SEQUENCE LISTING (42 sequences)

SEQ ID NO: 1 - DNA, 1300 bp, Abies koreana
tctcagattttgtcttttggttttgcgatttcggattagtaaatacattgcatgtgcgatcttcgaagaaggatgggctgcaaatcaggaacagactcggcgcagatcaatctgcccccaggtttcaggttctatcccacggacgaggagttgatggtgcattacttgtgtaaaagagcggcctcacagccctttccaattcctattattgcagagattgatttgtacaagttcgatccatggcaactacctgagaaggcgttatttggggagaaagaatggtacttcttcagtccacgagatagaaaatacccaaatggatcgcgtcccaaccgagctgcgggatctgggtattggaaagctactggaacggataaacctgttatggctggagctaacaggattaaggtcggagtgaagaaggctctggtgttttacaaagggaaggctcccaaaggcgtcaagacaaattggataatgcatgagtatcgtctcgccgatagtgctagtaaagcagcaaagagaaaaggaagcatacggttggatgactgggtactgtgtagaatatatgaaaagcattacgcatcgaaggccatgcctaaagaaagcaatgacttatcatcctgtgtagatcaagaggaagatcaatatatggtgaccgctgcatcaacatcccctccagacttggacgatagcaagttgctcctgaatctgcctcgtatcaattccctgaatagattaaactctcttctgaaattccccagcagggagatgatcaagataccagactcgaattataaccagacagaacaacgatacaattccaatgcagaaacggatgcaggttctaatgcagccggatgccaagcgatagctggttatgatcagaatccatcatactggagaagccagcagcaattcggaacctgctatgatcagatgatgcagaaccagtttcctcatcagttagagcatgtgacatccttagggaattctagtgctttttttgcgagcctggctgcagagctcctttgaagataatttatttcaaaatttctaactcgaatcaatctcttcctcccctgcaataatttgcacgggccacggactcccggcttcttaaatttttatgttcccatcattattatctacttcttttctacgccggttgctggttgtgtgctccagaatccatctattaccatgaaatccttctcttttgtattatattgttaaaaacatttttgaaggtatttccaaagtactttccttaattgattaattgattattagatctaag

SEQ ID NO: 2 - DNA, 1432 bp, Abies koreana
gatcataggatcaggagaaccacgatattggaaaaattgtgacgaaatttctttcaggatggtcggcctgtgtctatatatattctcatcttataattcttctgcgtaggggcaacgatttaagaaattgtagctggaagatagattgttgctgaatgtttgttggatcgatcaggttatagattagatcatgggtagaagccctagtagctggagctgttcaaaacatgaggagggtctcaatcgcgggggttggactgctagcgaggacaaggttttgagtgaatacattaaaactcatggagttgatcgatggacaactcttcccaagaaagcaggtctgaagcgaaatggtaaaagttgtagattgcgctggttgaactatcttcgtccagaaataaagcgaggaaatatttccccggaagaagaggagctcctcatcagattacatcgtcttcttggcaatcgatgggcattgatagcagggaggcttccaggccgaacgggcaatgaactaaagaattactggaattcccagttgagcaagagggtccccttcaagatgggtgaattcgaaaccaaatttcagaattccttaaagagaaagggcgtcccttctgactctaattacgacgaggaacgaatcgtaggcaaagataattatagctatcaaacagcagtgagatacagtggaagggcagatgtagacaatcttgagagttatagttatgatggtattaataaagcagcagatgatgatgttgtggaaatagaaatagatacctccaaatcatggtgccaattgctgatggagaattgtatgggagactatcagtacgatcgattggaagcggttaatgagcttcaatataagagcattatgagctctagcagtggaacagatgatcttcaccagtccacctcctcttccatcagtccaagtaaaacattgcaggaaaatcattcttcttcttcttgtaatttgttttaccaggatttcccggattttgattcctcatattttgatagcttcttagatccgagcatgctgtccagctccattcttccataggttatagcaggtcatggtgggattacatagtgccatgcgaatagaatttggtttatcaacagacataattaccatacccatgacttacaagtataagtagttttacaagtataccatacccatgacttatgacttagtacattaataagtgtattaagtggttaagtgtataaatagttaggcttttagttataaggtttagtttaattggtttgtgtaaagtttagtttaattagtttgtgtgatgaaatctggcagagttccggtcgatccgaaactttgttcgattttccgaacatcttggtcggtttaaaatggggcaaaactcccaaaggtagagttccatagtggaactatgcc

SEQ ID NO: 3 - DNA, 1396 bp, Abies koreana
tgaggatcatcttggtgtgaacatgccaaaacaggaaatcgccgccattaccgacttcagtgttaaagttccatccgcaacttccaccacatttctgccaactcattatgataatttgtcggcaccgactgaaggtcgttgtagcgcccgcgtggaaaaggtgagcgccgccgcctttttcgacccacagcatcacgagaacccatccccgcaattcatgagagctgactcaacatgtttatcacaattttcagattcatatcactttgcccaactacagcataatccagggttcacatatctttcacatttttcaaacccatatcagcatgcccaactgctgcataatccaggattaggtgccgaactggcaaattctcaccctacttctctccagcagtattcagctcatataagacctcagcagcagccagaaccttgtgatcatcagctgccttctatcacgtctatccaaaaccatccaagctttgattataatcgacaagtgcaatttacaaggagacctactttagcttcatcaatttatacaggtttcaggccatctatctccagtaattatgtgctccagcagcagcaagaacaacacatgcctattcagcaacaacgaactccatcaattagcaccacagggtatgaagagttatataacttgcgtggaagaattattgatcagacatctggcaacccgctggcttctgttcaatctcaatacgaatcctccgacaatgactataattcagtaggtcgtcgtgcatataattggcactgcaccgatctttgacgggagaaaggtcattcacaacagacgctagacttttctgggtgttcttctgtcggtaaaatttccaattttgatgtgcttcatgtttgaggacatccttataagctcttaatctaccacatggacgttatttatgtttatttatgttcaatcaacgatatatgccatagctagaaacacaagattttaactttgatcgcagtccataaacgcagtataataaccagcactacagaattctcaacaagtcgatttactggtaaactagctatttcagttttggtaatactgcgtgtcatgaaaacacagaagaacttattgttttgttcacatgaaatgtgacttttggagccactggtgtggtgaaatattacggccaagactggatgttgctgccaattttttatgcgtttgatcagcattatagatttggaattgctataggtttgctaatatccaaaatattttctgtaatcagtacaaatcatcacagcagtttcagatctttataagaaccattctatatattagaatatacagctcaaaaatagttaggatggtattacaagatccaggtttatacatttagaccag

SEQ ID NO: 4 - DNA, 1328 bp, Abies koreana
ctctgaaattgaggtcttcacagcttggggatttgataaatacctgtagacagatccaggatttgatccatctcatcccaagatggaaattctgtccgcttcttctctagaggggggttttaatgggaatcctgtgcttcaagggggtttcatgacgattcatccaaatgggcaaaacccagctagcagaaatgttgatggagatactataagcataatagacgaggcccatttggagctcccccttttcaataccactgaaacttcatggtattccagttcttctgaagacatagaggcatttttagataactgcgatatctctcatgcccataacctggcgccccagagtagtatgtacaggcccagtgcattcggctcatacttaagaccttatacccactctgtccattttaaaaatggcaaacagaatcaaatgaatagatgcttcaagtttctccgaaaaatcgacgagaagagaagagatgcgcagaggttaagtcaggctgccaatattcaagaagagcatgaattcgatgcatcttttccaaacaaggccgcttttcagcatatgatcgccgagcgtaacaggagagtcaaacttaaacaacatttctcgttactgcattctttcctacctcataattccaagagggacaaatattctgtactctcaaataccttcaagtatatgtgtgaattgaagcgccaagtggatgagctcgagcaaaaaaatcgtgccttggaagaatcgataaacctttcaaacaaagaggggtgtaacagttttgatcagagatttgaagaggatcaggcaacattagtttatagcaacgatgaagtcgttttggagcaatccaagaatatgcccaaccaagtaaacatgcgaataaatgttcagatcgacccactttccagcccaacgagtctcatcatcgtcctgctcgagcgtttaagagaattacaactggaggtcacaacagtccagtctaatatccaaacattcaagtttcaggctcatcttctcataacacaaattaagggtgatcagtgggagaatttcagatgggaaaatgttgcagaggttgtgagacgcacttttgctgtgaatgaatgaatgaaaaactcagtgaggatacacagaatttgccaatcagggactcagataatttgttataaaacgctcctgtataaattatatccaaccttctctctggagattggtgtctttctagttcttgtgatatctacaaatttcaacccttgatgggaaaatgatgtattcatcatttatatgtatatgtgagaggttcaattccattttattt

SEQ ID NO: 5 - DNA, 1681 bp, Abies koreana
aagcctagaaaacgcagggcgtaaggaataaagagaagaaagaaatagacaagcatagaaatcaatttctgtcaatcaatataggtagactagatgcaagaggtctcgagttcgattccatcagaagaagccttctgagagatggggagacaaccgtgctgtgacaagattggactgaagaaaggaccatggactgctgaggaggaccagaagctcatttctttcattctcaacaacggccattactgctggcgtgcggtcccgaagcttgctggcctgctcagatgtggcaagagctgcagattaaggtggacgaattacctacgcccggaccttaaacgcggagttttatcagaaaccgaagagcagctcattattgatctccattcccagctcggtaacaggtggtctaagattgctgcacacttacctggacggacagataatgagatcaagaattactggaacacgcacatcaagaaaaagatgaagcgcatgggaatcgatcctgtgacgcaccttccaataaccgaacaagccgagagcaaagcgagcaaggcattggatgaagcaacagaggattcagtatcagcgatcaaatcaggaacttccggccctactaggaaggcattattgcatgctgtgaccgaagtgtcatgtcgatcaaatgaatcagagataaccgaggaggccgaaaagagcattgatagcgtcaaggccgctaagattgaagctgcacagctcgcaacaggtgacgggttcgatcacatgcccatgctgttcgacgagagatcttcgacaatttatttgtatgatatgcctataactgagagccagaatttggaaaattcaagtatggatttcgtgggcaatgcagtgctaagaggcgcctcagacgatcagagcctactggaactcgcagaagatatgaccccagggattatttcagagcctttgaaatcccaagagggtttgtcatcaataaacgatgaactgccattaattaatccatgtccgtcttttaatgtcccgatcgaaacttcgacgagtacttcagcggatcaagaattgcaatggacaaacgaggtatccaacgcggcaactgctaattatgacatcttgctgggaggcgcgggttcgattcctgaaactgacatgcaattatggcatatcgagcatcttcaatcagcattcgacgcgggatgggatattcatcacagcgttaacaatgcctgcatattctccagtccactcttcttctaagaattctaattattatttttacaaaattaagctacatagatgagactttatgggttcaagcttcaaatcctgggcaccagggaatgaatttcccattcaaataggatttgcagttgttgtagtaatgtgtatatatctttaataataaatactgggatgtaatatttgctaggtttagtctaattgactggcctagcagcgggcattattattatgattcgtaggatatttgaatttaaagctggaagatctaggttgtctgcaatcaataaatttattagaatgctcgctttacatgtggcatttaaatgctttcattaattattatcatccatgaatgctctatcaaactgcatgcccatccaagtcttctgagacgtgaactgtaactgtaagacccatattcaataaatatctg

SEQ ID NO: 6 - DNA, 1763 bp, Abies koreana
gaagctgaatctgatcaggctggtgaaaaactttgttgcgtgaacagtttgatcgtaatggcgttttattattaaagatattaatggtattccccgtgcagatttgacctgggccaggcatggcatcatcgaaataataatatttttattctgagtccgcggggccggtattcaagtaaaatatcggttgtccgtgaggtgaaaatattgttgctgtttataaaaggctgggggagcttggtcgttcagtccttgctgtggctgtttgcagcattgcaattagtatctggagtccctcgttactcttgcagcgatttataagttaccatgtactggaaagcctgccaaggactggattcggctacctgatacaaaggggggttcacgagtccaacattttgaaaaggctaagaaactttgaaggttaaagaattggatgcaaactgggtcctcttagctacgattcatttgtatgcatggaccaaattacagacggctctcattccgcccgacatttgctttattctctgcagcaaccgcaagaacaacaatcagaggtgacattactgccaagcgatcataataacatgaatgcaatggacatgtgtatgcccgaccgacaaagaacaggacctaatccacagccgcagctgaattggccgcaacaacaacaattcttgagaacaatttctaattccgagcaaatgcacggtccgtttgtttcattgctcacatcttctgagatgtttgctgacggcattattacaagcgaatggcaacaattggcagaacaaaacggacatttattgacggtaaatccagaatacgccatgaacttaagcagatcaatgccgtctcaaggatctgcgctacctcgaacgctttgtcggtcattgtcctctcccaagggaccgctgaacttgcagagtgactggtcaaatttatccaataacacggaaagtatgtgtgctgtggagagcgagccaaatgccagaagcgcattaaaagcgccggcatgtttgggaccgtttccttcggacccgggctttgaagagcgcgcggcgaaattctcgtctcttaacggaaaaaactggaacgcaaaactgttaacatgtcaaagagcatcagtgggagaaacggagactggcagcttagaattcccttctgatgttgctcgaaacaaaccgcaagcggtctgttccgacgattccgaaagcaggaaggtgttttacagaacggcaagctgccctcctaccgtcgaaaatacggcagccgaaactgagaaatcgacggtcatggcggaagaatctgccgtctctgataagtcatctgcaagggacgccaagacttcagataacaatgccgtaaagaagaggaaaagcgtcaggacaagtgctcaaaaccacagggcaacagattccgaagagtccaaggagaagcgaagcaaatctgttcatcccagcgccaaggagaaagaggagataatgacgaaaacagagcagagtaacagtgacaattcagttgattccagtccaaaatctgcaagggaaaattcaaagcctttggagaaccagaagcaagactacattcatgtgagagctcggcgaggccaggcaaccgacagccatagcttagcagaaagagttagaagagagaaaattagtgcacgcatgaaatatttgcaggacttggttccggggtgcaataaagttacaggcagagcagtcatgcttgatgaaatcatcaactatgttcaatctctt

SEQ ID NO: 7 - DNA, 768 bp, Abies koreana
tcggaggacatggttttgtacgggtttctcaaggaggccgctgccaaggtattcctgcccccaacgcccaaggaagagctggtgacggagatgaagaaggaactgcgctttcccgtcatacgagagacggatgtctgtaaagaaacggcagccccgccgccgaagaaaccgatgacgaagaatgcggggaggcattaccggggcgtaaggcaacggccgtgggggaaatttgcggcggagataagggactccgcgcggcagggcgctagagtttggctcggcaccttcaatactgctgagcaagctgcgctcgcatatgaccgtgctgcttacaagatgaggggctccagggctttgcttaatttccccctcaatgttgggggagaggtgagtgatacttgtaataatacgtccaccttagaaaaggtggagagcaatggtgaaaataatttgagtaggaagagggaacgtgaggaagtgagtggtgaagtcgagttggataaacaatgtcgggtgagattggaagatgacaacaagatatgtcgaggaatggttgaagagcttctttgtgggaagatggcaacaccaccagagacaccaagtggcatagggcaactcatggtgaattgaggtttctttgagttatttcattctctttccaaaaaattatcattctttctatgtgtcaaaaaattgtaactttcattttcgatgatgatgatggtggtaaacatccatttagatcaaactttggtctatcgaacctaa

SEQ ID NO: 8 - DNA, 2569 bp, Abies koreana
cggaattttcatagaaaaaaagagggaacgttctggaaacggcgaaaccgcctaagtaactccagacgttcgtcaagaactctccagaaaccctgaaatcatcccaacgcttccagagacatctaaaaccctttctctatcttctagtgtcggaaccctatcatttaaattggctgtttggcttcgcaatctctcgcaggaacacgcgaggaagtcttttgtgcagtttaatacgcattctataaggcgcgcgctagggattttgatattgggtcgagtgtttggatatcgaatcgagaatttctggtgctgctataggtttgggtttgggggtttagaattccctttgtagttatctggttccctattctagacgattagaattccaatggcggacgttaacatggagagcggggaggtggagacctttgctttccaggctgaaattaatcagttgctcagtctcatcatcaacactttttacagtaacaaggagatttttctcagggagctcatcagcaattcatctgatgcgctggacaaaatccggtttgaaagtctgacggataagagcaagctggatgcgcagccggagctcttcattcggattgttcccgacaaggctaacaagagtctcaccattattgacagcggaataggcatgaccaaagccgatttggtgaacaacttgggaacaattgcaaggtcggggaccaaggagttcatggaggctttacaagccggggcggatgtgagcatgattggtcagttcggtgtcggtttctactcggcctacctcgtcgctgagaaggtgattgttaccaccaagcacaacgatgatgatcaatatatttgggaatctcaggctggagggtcgtttactgttaccagagacgtgaatggggagcccctgggcaggggaactaaaatcactctgtttctcaaggaagaccagctggagtacttggaggagcggcggttgaaggatctggtcaagaagcattctgagtttattagctaccccatctatttgtggactgagaaaaccaccgaaaaggaggtgagcgatgacgaagaggagggtgaagagaagaagaaggaagttgaggaaggggatattgaggaagtggaggaggacaaggagaaggataagaagaaaaagaaggtgaaggaagtgtcccatgaatggaatctggtgaacaagcagaagcccatttggatgaggaagccagaggaaattagcaaggaagaatacacgtccttctacaagagcctcaccaatgactgggaggaccacttggctgtgaaacatttctctgtggagggtcaattggagttcaaggctgttctctttgttcctaagagggctccgttcgatctgttcgatactaggaagaagctcaacaacattaagctttatgtgaggagggtgttcataatggacaattgtgaggaactcattcctgaatacctaggttttatcaaaggtgtggtggactccgatgatctgccccttaacatctctcgggagatgctgcagcaaaacaagatcctgaaggtaatcaggaaaaatttggtgaagaaatgtattgagatgttcaacgagatcgccgagaacaaagaggactatgcgaagttctatgaggcattttcgaagaacttaaaattgggtatacacgaggacagccagaacagggctaagctcgcagatttgctaaggtatcactccacgaagagtggagacgagatgacgagtctgaaggactatgtgacccgaatgaaagaggggcagaaggatatctattacatcacaggtgagagcaagaaggcggtggagaattctccgttcctggagagactgaagaggaagggttatgaagtcctgttcatggtggatgccatcgatgagtacgccatagggcagttgaaggagtacgacgggaagaagctggtgtccgccacgaaagaagggctgaagctggaggagacggaggaggagaagaagaagaaggaagagaagaaagctgcttttgagagcctgtgcaaagtgatgaaggaaatcttgggcgacaaggtggagaaggtagtggtatcggatcgcattgtggactctccgtgctgtctggtaacaggggagtatggatggacggcgaacatggagagaataatgaaagcccaagccctccgcgacagctctatgtcgtcctacatgtccagcaagaagacaatggagataaacccggagaatgccataatggaggagctaaggaagagggccgaggccgacaagcacgacaaagcggtgaaggatctggtcatgctcttgtatgagaccgctctgctcacttcggggttcagcctggaggagccgaacacatttgccagcagaatccataggatgcttaagctcggcctcagcatcgaggacgacgccaccgctgagttggacgcagatatgccgcccttggaagacgacgccgctgtccaggaggagagcaagatggaggaggtcgactaagtcgcaccttgactctataaggaagaaatcaaattttctgagagggatttc

SEQ ID NO: 9 - DNA, 2688 bp, Abies koreana
gccgtggaagcttctgtagtgaaacgggagaaatctctataattccagagaattctctagacacctgtccatccatggcgttataggctgtcaccgacttccatgtttaaaaggacctctcgatacggttcatttcattccaattcatcaccgttgcgttgctaaattggcaaggaatacagaacgtcttgcttgacttctctttgtttttgtttcactgtgtattcttgaggattttgctgagcttttgagttttaacaatggcaagcggcgaaggtaaggccatcggaatcgatctgggaaccacatacagttgcgtgggagtttggcagcacgacagagtcgaaataattcccaatgaccaaggtaacagaacgactccttcttatgttgcttttactgatacagagcgcttaattggggacgcagcgaagaatcaagtggccatgaatccccaaaatacggtcttcgatgccaagagactcatcggcaggcggttcagcgatgcttctgtgcaaaacgacatgaagctttggcccttccgggtcttttctggccccggcgataagcctatgatcgaggttggctacaagggcgagaagaagcagttttctcccgaggaaatttcttccatggtgctcaccaagatgaaggagattgcggaggcttttcttggccagactgtgaagaacgctgttgttactgttcctgcctatttcaatgactcgcagagacaggcgaccaaggacgctggacttattgctgggctgaatgtgctgagaatcattaatgagcccactgctgctgctattgcttatggacttgataagaagtccagctctaccaaagaaaagaatgtgctaatatttgatttgggaggaggaacgtttgatgtctcgattttgacaattgaagaggggatcttcgaagtaaaggctactgctggagatacccacttgggaggtgaggattttgataacagaatggtgaaccactttgtgcaagagttcaagagaaagcataggaaggacatcagtggaaatgctagagctcttcgcaggcttcgaacatcatgtgagagagcaaaaagaacgctgtcgtctactacacagacaacgattgagattgattccctgtatgaaggaatcgatttctatgccaccattaccagagctcgcttcgaggaactcaacatggacctgttcaggcggtgtatggagcccgtggagaaatgcatgcgggacgccaagatggacaaaacccaaatagacgacgttgtcttggtcgggggttcgactcgcatccccaaagtgcagcaattactccaagatttcttcaatggcaaagagctatgcaagagcattaacccagacgaagccgtggcctatggcgcagctgtccaagccgccattctgagcggcgaaggcaacgagaaggtccaggacctgctgttactggatgtcacacctctaagtcttggtcttgaaactgcgggaggcgtaatgacagttctaataccgcggaataccaccattcccaccaagaaggagcagattttctcaacctattctgataatcagcctggggttttaattcaagtatatgaaggcgaacggacgcggacgaaggacaataacctgctgggcaaattcgagctcgccggaatcccaccggctcctagaggagtcccgcagattaatgtctgcttcgacatagacgccaatggcatcctcaatgtttcagccgaagacaaaaccgcaggagtgaagaacaagatcaccatcaccaatgacaagggccggctgagcaaggaggacatcgagaaaatggtccacgatgctgagaagtacaagtccgaggatgaggaggtcaggaaaaaggtggatgccaagaattcccttgaaaactatgcctataatatgaggaacactgttagagacgagaagttttcaggtaaacttgatccggctgaaaagtccaagattgaagaggccatcaatggtgctctcagctggcttgatcaaaaccaattggctgaggttgatgagtttgaagacaagatgaaggaattggagggcatctgcaatcccatcatcgctaagatgtaccagggtggggctgctcccatgggcgctgatgatgatattcctacctctggtgggggtgctgctggttctcgtggtccaggccccaagattgaagaagttgactaaactacatattttctctctctatctttattatattaatatgtaattgattactttattatatatacatataggtagatagttatatattttgagttagtagtgcatcattgtgaaaattgcattttcagagatgtttatgtaaattgcattgcatttgaaagagagtcggtattgccgccttccctcttccctgtgtgtgtgaagggttcagttttaataaaataccgagaattgttcccaaaacagcttttttcaaagcatggaattgtaatgaatgtcattgctcctctttctatttccaagatcttgtctcattttgtgatagctagtcttctggactccttaaagtggtgcaggatccacaatgatgtgttctaaacggcatatcagaatatcaattggttatggattggcagttttctatattctaaaagagttgtagcataatttgtgttttgttaacagttggagagat

SEQ ID NO: 10 - DNA, 1246 bp, Abies koreana
cgccgactggggattattttgttcagtagccccgacatcaagtctcgccggcctaattcggttcaaatcggtttgcttgcagctctgagcgatttgctatgatggcagataaccagcgagtctatccgtcgacggcagacttagagaagaacgcaggcgaatatggaaacgagaagcccagcgctccgttggtgcccaattcgagcagtatttcgtctgagaggggtcgagtcggtcctgttcctgttcctcattcgggcgggcagatgaaagtacagtttccaaaggaccaagtcgtctccagacgagagcttcccagatactactcaccgcctcccaagcgccggaactcctgttgccgctgcctgtgctgcacgatttgctttctaatcggtttcgtcgtgacaattgccatcgtctgtggtattctgtttttgatcttccggccgaagatccccaaatactccgtcgacagcatccacattactaacttcagcgtcaacgccgacctcagtacaaattgccagttttccgtgaagataagggcccggaatcccaacaacaagatcggcatatattatctcgaccacagccatctcgccgttttgtactccggcactgaactctgcacgggcgccctgccggtgttctaccagggccacaagaacaccagtttcatggacgtggcgctcactgggaccggcgctccgctcaccagcgaaatggtttccacgcttaaagcgcagcagcagcagggcagcattcccctgaatttgaaggccaacgttcccgtgaagataaaattggggaaattgaagtcgatgaagattaaggtccgcgtgcgatgggacctgatcgtggacaagctcgatgctactgccaaagttacagagaagtcgtccaaggtgagggtgaagctgtgaagattgcgagatcgatctatggggggttttgttgcgcatcttcggatgtattgaatacatggttagggcgaggatagggctacatgttttctttgatttgatcatataaatgctacggctgtagctagatttttcgatatacatttttttttgttcatactccatatactacattaatggccgcccttcaggtggccattgtttatatgtagacatattactgtatttaataaatatatgtgcattctttactctcttttgttgtttacttcttctgttctttcttttggattagtggaaaactgttttccaatccttggtag

SEQ ID NO: 11 - DNA, 953 bp, Abies koreana
caggtttctagagatatgatgactaggagaaacgcatcgatgcttccacacaattctcccctccttacttaaatgccacgcctccccgctttttcagacatcgagtgtgagacatcggcaattgacaggatttgatttactgtgcgtttttctctgccaaagcaacaaagatcttgtgttaccagagacccttggaaggaagaatgaacgatttggttagagatatggagcatctgctcgacatgtccgacgaaggggagaaggatctgcagcaacctgcgcgcagatacgttcgagaaacaagggccatgtttagaaccaccgctgatattttggaggctcccgatagctatatcttcgttctcgacatgcccggcctcgagactaataatataaaggtgaaagtagagaacaatatccttcacatcgcggggaagaagaagaagaaaaagcagctgaatccggaggtgaaggtgataagattggagaggcggagggcgaggtacatgaggaaatttagtctgcccggagactccaatttagaggacataaaggctacttacaaggacggcgttttgactgtcaccgtggccaagcgccagacctccgaggccgagaaacctaagtctgtcaccattcccatttcttgaaccctatccctaaatcaaagaatacttctcagctgaactctttctttctttacagtttcagttatctattttccccaacgtattaagctcgtgcacagagagagcagagagcattgtgttggtaaatatttgtattgtatgtgatgtgagttatactgactttcataataacacaggagttgtgttttgtatgctttgtttgcccttcaaaatagtaattgcctggctatttatgtctgttaaatggccgtggattattcgagccgtttaagattatggatgcatgtttgagaattcgcccag

SEQ ID NO: 12 - DNA, 1698 bp, Abies koreana
gaggggcatgttaagaccctacatgaatatttatgtgttgtataaggaggttgtcccctcaaagtggcttactcttattgatatttttatcatattcaaggcgcttcacttctgatttttatgtttttatgctatttgggaatttattgcataacattggcttatcacttgtggaatttttagctagaggtattcccaccatatggagtctctatcatttaagttgaagatatcaaccttggccttttagcttctgaagtattcacgggacttgaaatatttcttcagtcccatgagccatgatttaaccacttgtcatgtcttaccttctccatcaaatgttggtggtatcgtcttcttaaattctccatatggtttctctttgaaggacttcctactcttggatcctttggatgagtaatatgagattcttgatgcattcttgttatcactgaagttattgtcaccaaattgtccagtcatccaaatacacctctaagaacttataaataaagtccttgaatgttgtcaccactactctaaaaaatattgttggtgcattcttcaaccaaaaaggcatcactgtgtattggtataagcccattcaataacaaatgcctttttgtatctatcctccttttctatattaatttgatgataacccgagaatccatcagtaaagagtatgcctcttggccccccacattctctaatcctttgtttgtgaatggagggggaaaatggtcattgacacaagcatcattcaacttcctatggtccacacacagtctaatgcctctagtcttcttatcttgtataactacacgaatgatccattttgattcttccactggttctatcaatcatccttcaagcatcttatcaatttcctccctcactttctatttgtacttagagtttaatctataaggactctgctttgttggccttgcatcttccttcaaatgtaccttcatccccccaactctccttctatgcctatcatttcagagaatgaggtgggaaataagtcatcatattccttcggcatatcagcggcctaattcactatttcatcatcccaatagtcgcctatgagtgcaatcttgggtgtcttttttgcgccaatgttgaccttattagtcttgataggccttacatatttaggtgcatctacatctagccctttatctttaatcaacatatgtccttcaatttcctggatgttgatttttctgagctatgagttgccaaaacccacagagaggcacacctgaaattaaaggaaaaatatgggtccaaacccacagagagccaagctccacagggaaccgaactggagagaaacttccaaattgagaaaactcataaaaatcccaatgcacagaagaaaccaaacctaaaagaaacgcaagggtgccctcatcctttataggcatgagcgtcattcttccacaccgagtgatgatgtgccagctcctcaaccaaccattatgcgcaagggtcttcatcatgacaaaagctggagatttgggttccaatgccttcaaccctgtacgcacctcgaattgtcattgtcacaccgagctcaagcattcgagggcttgcaatagggattgaatccaaatgagcattcacgggaggagcacgtgttgtatttaacaaagttgttacgggggatgaggtgttagcacgggc

SEQ ID NO: 13 - DNA, 2259 bp, Abies koreana
gccttaactctgcagctgtggctactgaatatagtattgtatttgagtgagggtttttattttatattgtagatagataaacagtcaaattttttgtttggtagctgatccaaatcaagatggcgtcgcacaagcccaagtcgaagcagggacaggcgatcggaatcgatctcggaaccacgtacagctgcgtagctgtatggcaacgcgatcgagtggaaatcatcgccaacgatcagggcaatcgcaccactccttcctttgttgctttcactgacactgaacgccttattggcgaggctgctaaaaatcaagccgccatgaatcccgtaaatacagtatatgatgcaaagcgattgatcggccggaagttcagcgatccagtcgtgcaagcagacatgaaaatgtggccattcaaggtggtggctggacaaggcgataagcccttcatcgcggtggaatacaaaggagaaccaaagaaattcgccccagaagagatatcgtccatggtgttaacaaagatgaaggaaacggccgagacctacatgggcacaacggtgaatgacgcagtcgtcactgttcccgcgtacttcaatgattctcagaggcaagccacgaaggacgccggagccattgctggattaaacgtgatacgaatcatcaacgaaccaacggcggctgcaatagcctacgggctggacgacaagagcaatatctcggggagcttcagagaaaaaaacatactcatattcgatctgggtggaggaaccttcgatgtatcggtcttggccgtggtaggcgggctcttcgaggtgaaggccacggcaggggacacccacctggggggagaggacttcgataacagaatggtgaatcattttgcacaggaattcaagaggagacacagaaaggatatcaccaccaacgccaaggcgctgaggcgccttaaaacagcctgcgagagagccaagaggacgctgtccacgaccacgcagacggccatagaaatcgattccttgcacgacggaatagacttctatgccaagatgaccagagccagattcgaggaactcaacatggatctgttcaggaaatgcatggaccccgttgagaaatgcctcagggacgccaagatggataaatcgcagataaacgacgttgtgctggtaggaggatccaccaggattcccaaaatccagaagcttttgcaggaccttttcaatgggaaggagctttgcaagagcattaatccggacgaggctgttgcctatggtgcggcggtgcaggccgcgattctgagtggagaaacgaatgagaaagtgcaggatgttttgctgctggatgtggcacctttgagtctagggctggagactgcaggagggataatgactgttctaatccctcgcaacactacaattcctgctaaaaaagagaaggtttttactacttatgcagacaaccagacttcggttctgattcaggtctacgaaggtgaaagggcccggagcagggataataatcttcttggcaagtttgtcttgtctggaatccctcctgcaccaagaggggttcctcagatcaatgtctgttttgatatcgatgccaacggaattctcaacgtcagtgcagaggacaagactgcagggatcaggaacaagatcaccataactaatgataagggtagactgagcaaagcagagattgagaaaatggtgcatgaagcagaaaagtacaagcaagaagatgaggatgtgaagaggaggtctgatgctaggaatacattggagaactatacttataacatgaggactactgtcaaggatgacaggtttggtaagaagcttagctcttctgacaaggggaagattgagaaggccatccaaggtgcgatagattggctggagaaaaatcagcttgcagaggttgatgaatacgaggataaattgaaggagcttgaacgcctctgtaatcccatcatctctaaggcatatggaaattctggttattataacagcaatcctgacgggccaaaggtcgaagaggttgattagatttgtcccaaagtaatgttgagctgagttgctcgagctctatagtgttagaggattattactagctgtgatctggtcttacagtacttacttcaatgctgtgtaaagatacagtacgattatatgttatatagcatataagttttccaggaggaacttcgttcacttaggtttatggatttaaattaattatttagagttatttgag

SEQ ID NO: 14 - DNA, 2505 bp, Abies koreana
ctgatataccccgcactaattaaacactagctgcgattgcctgcctctgcttcacccagagcctttgtcgtctatcattatggcaataacaagcagagcaggagttgcacccgtccttcaagccgactgccattcgactggccatttccatctaattacagagttgggttctcgtaatagtgccatgtttcaatcggcaccatttcaacagattagagtaaccactaaaagaaagcgctgtatcttgtctgccaagctaattaattcggacggagaaaaaaatgaaaaatgctagtgcagctgtggagattgcttcaaagaatgacttcccagctgagaaacctcccacgccgttgcttgacaccatcaattatccaaaacatctaaagaatctctctatacgggatctggagcaactagccacagaaattagagatgaacttgtgttcggagtgtcaaaaactggaggccacctgggaggtagcctcggtgttgtagatctaaccgtggctcttcatcacgtctttgacgcacccgaggataagattatatgggatgtaggtcatcaggcatatccacacaagatcctgacaggtagaaggtccaaaatgcatacgatcagacagacatctggcttggctggatttcccaaacgagacgaaagcaagtacgatgccttcggcgctggacacagttctaccagtatttccgctggactcggaatggcagttgggagagatttattgaggaaaggtaaccacgtcgtggccgtgattggggacggagctatgacagcaggacaagcatacgaggcgatgaataattcaggatatctggaatccaatctcatcataattcttaacgataataaacaggtttctctcccgactgctaccctagacggagccgcgccgccagtaggagcactcaccagggctcttacaaagctccagtccagcaaaaagcttcgcaaactgagagaagctgccaagggtctcacgaaacagattggcggccaaactcatgaaatcgcatcaaaagttgatgagtacgcaagagggatgataagcccagcaggttcctcgctgtttgatgagctgggtctttattacatcggacctgtagatggccacaacattgaagacatggtgacaatcttagagaagattaaaagcatgcctgctagcggaccagtgctgattcacttggtgacagagaaaggaaaggggtaccctccagcggagaaagctgctgacaaattacatggtgtggtgaagttcgatccatctacaggcaagcaattcaaatccaaaagttcggtgctaagctatacacaatacttcgctgaagctctgatagcggaggccgaagtagacagcaaaattgtggctatccatgctgccatgggtggaggcacaggcctcaattatttccaaaagaggtttcctgaacgctgcttcgatgtcggaattgcagagcaacatgcggtcacatttgctgcaggcctggctacggaaggccttaaaccgttctgtgctatttactccacttttctgcagcgaggttacgaccaggtggttcacgacgtggatctgcaaaagctgccggtgcgttttgcaatggatcgggctggactagttggggcagacggccctacccattgtgggtcttttgatgtggcttatatggcctgtttgccaaacatggttgttatggctccttccgacgaggtagaactcatgcacatggttgcaactgctgcggcaattgacgatcgacccagctgtttcaggttccctaggggcaacggagtaggcttgtcaaatttgcccctaaaaaacaaaggtcttccacttgagatcggcagaggccgcattctggtagaaggcacccgagttgcgattctagggtttggtacgatagtacaaaactgtctttcagctggaaaaatactaaatgaacaagctggtatatcagtcaccatagcagacgctcgtttctgcaagcctttggatggagatttgatcaaacgattggccaaagagcacgaaatcctgttgacagtggaggaaggatccattgggggttttggatctcatgtctcccatttcctggctttgaacgggctgcttgacggaaagctgaagtggagggctatggtactaccagatagatacattgaccatggagctccacaagatcaaattgaggaagcagggttgtctccgacgcacattgcggctaccataatgtccctcctgggcaagcctcacgatgctctacttaagcaccgataactacgccatcgacctctattttcaggcgacaagcgtctgggaacatagtttgcttatcatttcaataaggccacacaagaaggctgatcagccaataaaacgtgaaaaggcaaaggccgcttttatttatcaaatgtaaaacagtttatcaaatgtaaaaacagtttgaattaaatatgataataacaattatatccttttcaattac

SEQ ID NO: 15 - DNA, 20 bp, Artificial Sequence
  primer
tggctgcagagctcctttga

SEQ ID NO: 16 - DNA, 20 bp, Artificial Sequence
  primer
tctggagcacacaaccagca

SEQ ID NO: 17 - DNA, 20 bp, Artificial Sequence
  primer
aggatggtcggcctgtgtct

SEQ ID NO: 18 - DNA, 20 bp, Artificial Sequence
  primer
caacccccgcgattgagacc

SEQ ID NO: 19 - DNA, 20 bp, Artificial Sequence
  primer
tcgccgccattaccgacttc

SEQ ID NO: 20 - DNA, 20 bp, Artificial Sequence
  primer
attgcggggatgggttctcg

SEQ ID NO: 21 - DNA, 20 bp, Artificial Sequence
  primer
cgccgagcgtaacaggagag

SEQ ID NO: 22 - DNA, 20 bp, Artificial Sequence
  primer
tcgagctcatccacttggcg

SEQ ID NO: 23 - DNA, 20 bp, Artificial Sequence
  primer
ccaacgcggcaactgctaat

SEQ ID NO: 24 - DNA, 20 bp, Artificial Sequence
  primer
atcccgcgtcgaatgctgat

SEQ ID NO: 25 - DNA, 20 bp, Artificial Sequence
  primer
agcggtctgttccgacgatt

SEQ ID NO: 26 - DNA, 20 bp, Artificial Sequence
  primer
ccgccatgaccgtcgatttc

SEQ ID NO: 27 - DNA, 20 bp, Artificial Sequence
  primer
ccgccgaagaaaccgatgac

SEQ ID NO: 28 - DNA, 20 bp, Artificial Sequence
  primer
aaggtgccgagccaaactct

SEQ ID NO: 29 - DNA, 20 bp, Artificial Sequence
  primer
acgtcagtcctcccaaggtg

SEQ ID NO: 30 - DNA, 20 bp, Artificial Sequence
  primer
cattggcccgcagtgacttg

SEQ ID NO: 31 - DNA, 20 bp, Artificial Sequence
  primer
tgtccaagccgccattctga

SEQ ID NO: 32 - DNA, 20 bp, Artificial Sequence
  primer
tcattacgcctcccgcagtt

SEQ ID NO: 33 - DNA, 20 bp, Artificial Sequence
  primer
ccgttggtgcccaattcgag

SEQ ID NO: 34 - DNA, 20 bp, Artificial Sequence
  primer
caaatcgtgcagcacaggca

SEQ ID NO: 35 - DNA, 20 bp, Artificial Sequence
  primer
agcagctgaatccggaggtg

SEQ ID NO: 36 - DNA, 21 bp, Artificial Sequence
  primer
cttaggtttctcggcctcgga

SEQ ID NO: 37 - DNA, 20 bp, Artificial Sequence
  primer
agagccaagctccacaggga

SEQ ID NO: 38 - DNA, 20 bp, Artificial Sequence
  primer
gagggcacccttgcgtttct

SEQ ID NO: 39 - DNA, 20 bp, Artificial Sequence
  primer
agctgcgtagctgtatggca

SEQ ID NO: 40 - DNA, 20 bp, Artificial Sequence
  primer
tacgggattcatggcggctt

SEQ ID NO: 41 - DNA, 20 bp, Artificial Sequence
  primer
ggctccttccgacgaggtag

SEQ ID NO: 42 - DNA, 20 bp, Artificial Sequence
  primer
ggcctctgccgatctcaagt

(19) United States
(12) Patent Application Publication
(10) Pub. No.: US 2019/0309375 A1
(43) Pub. Date: Oct. 10, 2019

(54) AN ISOLATED GENE EXPRESSED IN RESPONSE TO HEAT TREATMENT IN KOREAN FIR OF ABIES GENUS

(71) Applicant: NATIONAL INSTITUTE OF ECOLOGY, Chungcheongnam-do (KR)

(72) Inventors: Hyeong Cheol PARK, Gunsan-si Jeollabuk-do (KR); Jung Eun HWANG, Jeongeup-si Jeollabuk-do (KR)

(21) Appl. No.: 16/323,946

(22) PCT Filed: Jun. 16, 2017
(86) PCT No.: PCT/KR2017/006322

§ 371 Date: Feb. 7, 2019

(51) Int. Cl. C12Q 1/6895; G16B 10/00; G16B 20/40

(52) U.S. Cl. CPC C12Q 1/6895; G16B 10/00; C12Q 2600/13; C12Q 2600/158; G16B 20/40

(57) ABSTRACT

The present invention relates to a genome-wide analysis of gene expression levels of the Korean fir of Abies genus. More specifically, the present invention relates to the isolated genes expressed in response to heat treatment using a next generation sequencing-based platform.

### SEQUENCE LISTING

The instant application contains a Sequence Listing which has been submitted electronically in ASCII format and is hereby incorporated by reference in its entirety. Said ASCII copy, created on 6 Feb. 2019, is named MK-235_NE_US_Sequence listing.txt and is 37.8 kilobytes in size.

### TECHNICAL FIELD

The present invention relates to a genome-wide analysis of gene expression levels of the Korean fir of Abies genus. More specifically, the present invention relates to an isolated gene expressed in response to heat treatment using a next generation sequencing-based platform.

### DESCRIPTION OF PRIOR ART

Korean fir (Abies koreana) is a valuable tree species for ornamental purposes, which is an endemic but rare species in Korea. It has grown in the upper regions of Mt. Halla, Mt. Chiri, Mt. Mudung, Mt. Kaji and Mt. Duckyu, located in the southern part of the Korean peninsula. Recently, the Korean fir populations have undergone a large dieback, resulting in a severe decline. This dieback can be presumed to be the result of complex interactions among multiple environmental factors caused by global warming.

Since this species is susceptible to climate changes, it has been designated as an indicator species for detecting climate change by the Korean Government. In case of the ordinary trees, the tolerance against high temperatures remains largely unstudied. Thus, it is essential to reveal the molecular response mechanisms of species vulnerable to heat stress, which will aid in understanding the heat tolerance of Korean fir.

High temperatures can be a cause of growth and development reduction, which may become a major issue in the coming years owing to global warming. Global temperatures are predicted to be raised by an additional 2-6° C. by the end of 21st century. Plants can respond to high temperatures by altering the expression levels of thousands of genes, followed by the change of cellular, physiological, and biochemical processes. However, there have been some differences in responses to heat stress among various species and genotypes. For the vast majority of species, transcriptomes have been still largely uncharacterised. Even in species whose substantial informations are available, it may be the form of partially sequenced transcriptomes.

Upon exposure to stress, various genes have been induced to make a function, which enables the plant to respond the abiotic stressors. There are several transcriptional regulatory networks involved in stress-induced changes in gene expression.

Stress-induced genes can up-regulate the expression levels of a plurality of downstream genes that provide an abiotic stress tolerance to extremely high temperature, severe drought and high salinity. Thus, the analysis of gene expression levels can be a valuable tool in understanding the transcriptome dynamics and the potential for manipulating gene expression patterns in plants.

Until now, microarrays based on either cDNAs or, in the case of model organisms, oligonucleotides have been the main tools for assessing global patterns of gene expression. According to the development of a high-throughput sequencing technology, RNA sequencing (RNA-seq) has been successfully applied for gene expression profilings and other transcriptome studies in many plants, including Arabiodopsis, rice, and poplar.

Such sequencing-based method can detect the absolute expression levels, rather than relative gene expression changes, which requires to overcome many of the inherent limitations of microarray-based systems. In the past, it has been considered that the de novo assembly of very short-read sequences is difficult without a known reference.

According to the recent development and optimization of a de novo short-read assembly method, now it allows for the cost-effective assembly of transcriptomes of non-model organisms with unknown genomes, opening the door for performing numerous and substantial new analysis. Therefore, this method has made it possible to sequence the transcriptomes of species lacking a sequenced genome, such as Picea abies. However, no comparative transcriptomic analysis have been performed using next-generation sequencing technologies in the Abies genus under an environmental stress simulation.

In the present invention, the inventors have performed a genome-wide analysis of gene expression levels of the Korean fir of Abies genus. Finally, 14 important genes expressed in response to heat treatment have been isolated and sequenced using a next generation sequencing-based Illumina paired-end platform. Therefore, the present invention has been completed by isolating and identifying 14 important genes, which can be used to create a reference transcriptome expressed under the heat treatment.

### Problem to be Solved

The technical problem to be solved is to perform a genome-wide analysis of gene expression levels of the Korean fir of Abies genus. Further, the present invention is intended to isolate and identify the important genes, which can be used to create a reference transcriptome expressed under the heat treatment.

### Means for Solving the Problem

The object of present invention is to provide an isolated gene expressed in response to heat treatment of the Korean fir of Abies genus, wherein the expression of an isolated gene of c142609_g1_i1 (NAC) (SEQ ID NO: 1); c207159_g1_i1 (MYB) (SEQ ID NO: 2); c124199_g1_i1 (ERF) (SEQ ID NO: 3); and c173884_g1_i1 (bHLH) (SEQ ID NO: 4) have been up-regulated, the expression of an isolated gene of c85122_g1_i1 (MYB) (SEQ ID NO: 5); c199182_g1_i2 (bHLH) (SEQ ID NO: 6); and c189548_g3_i1 (ERF) (SEQ ID NO: 7) have been down-regulated.

The other object of present invention is to provide an isolated gene that encoded HSP (heat shock protein) expressed in response to heat treatment of the Korean fir of Abies genus, wherein the expression of an isolated gene of c217843_g2_i1 (Hsp90) (SEQ ID NO: 8); c149565_g1_i1 (Hsp70) (SEQ ID NO: 9); c199303_g3_i1 (Hsp60) (SEQ ID NO: 10); and c156586_g1_i1 (sHsp) (SEQ ID NO: 11) have been up-regulated, the expression of an isolated gene of c205143_g5_i1 (Hsp90) (SEQ ID NO: 12); c149639_g1_i1 (Hsp70) (SEQ ID NO: 13); and c202543_g1_i1 (Hsp70) (SEQ ID NO: 14) have been down-regulated.

Said isolated genes expressed in response to heat treatment of the Korean fir of Abies genus has been isolated, wherein a gene of c142609_g1_i1 (NAC) (SEQ ID NO: 1) has been isolated using the primer pair set of SEQ ID NO: 15 and SEQ ID NO: 16, a gene of c207159_g1_i1 (MYB) (SEQ ID NO: 2); has been isolated using the primer pair set of SEQ ID NO: 17 and SEQ ID NO: 18, a gene of c124199_g1_i1 (ERF) (SEQ ID NO: 3) has been isolated using the primer pair set of SEQ ID NO: 19 and SEQ ID NO: 20, a gene of c173884_g1_i1 (bHLH) (SEQ ID NO: 4) has been isolated using the primer pair set of SEQ ID NO: 21 and SEQ ID NO: 22, a gene of c85122_g1_i1 (MYB) (SEQ ID NO: 5) has been isolated using the primer pair set of SEQ ID NO: 23 and SEQ ID NO: 24, a gene of c199182_g1_i2 (bHLH) (SEQ ID NO: 6) has been isolated using the primer pair set of SEQ ID NO: 25 and SEQ ID NO: 26 and a gene of c189548_g3_i1 (ERF) (SEQ ID NO: 7) has been isolated using the primer pair set of SEQ ID NO: 27 and SEQ ID NO: 28.

Said isolated genes that encoded HSP (heat shock protein) expressed in response to heat treatment of the Korean fir of Abies genus has been isolated, wherein a gene of c217843_g2_i1 (Hsp90) (SEQ ID NO: 8) has been isolated using the primer pair set of SEQ ID NO: 29 and SEQ ID NO: 30, a gene of c149565_g1_i1 (Hsp70) (SEQ ID NO: 9) has been isolated using the primer pair set of SEQ ID NO: 31 and SEQ ID NO: 32, a gene of c199303_g3_i1 (Hsp60) (SEQ ID NO: 10) has been isolated using the primer pair set of SEQ ID NO: 33 and SEQ ID NO: 34, a gene of c156586_g1_i1 (sHsp) (SEQ ID NO: 11) has been isolated using the primer pair set of SEQ ID NO: 35 and SEQ ID NO: 36, a gene of c205143_g5_i1 (Hsp90) (SEQ ID NO: 12) has been isolated using the primer pair set of SEQ ID NO: 37 and SEQ ID NO: 38, a gene of c149639_g1_i1 (Hsp70) (SEQ ID NO: 13) has been isolated using the primer pair set of SEQ ID NO: 39 and SEQ ID NO: 40 and a gene of c202543_g1_i1 (Hsp70) (SEQ ID NO: 14) has been isolated using the primer pair set of SEQ ID NO: 41 and SEQ ID NO: 42.

### Advantageous Effect

The advantageous effects of the present invention is to afford a genome-wide analysis of gene expression levels of the Korean fir of Abies genus. Further, the present invention is to provide the isolated and identified 14 genes, which can be used to create a reference transcriptome expressed under the heat treatment.

### BRIEF DESCRIPTION OF DRAWINGS

FIG. 1 indicates a Gene Ontology (GO) classification of Abies koreana transcripts. A total of 406,207 transcripts were functionally classified into 3 main functional categories: biological processes (FIG. 1a), molecular functions (FIG. 1b), and cellular components (FIG. 1c). The right y-axis indicates the number of transcripts.

FIG. 2 indicates distribution of differentially expressed Abies koreana transcripts in heat-treated samples compared with control conditions. (FIG. 2a) Distributions of up-regulated and down-regulated transcripts. The up-regulated and down-regulated transcripts indicate log 2>1 and log 2<1 of twofold values in comparison with under control conditions. (FIG. 2b) Scatter plot of the normalised expression levels of all transcripts under control and heat-treated conditions. Each point represents the mean expression level of a gene under control and heat-treated conditions.

FIG. 3 indicates family distribution of the transcription factors in the Korean fir transcriptome. (FIG. 3a) The numbers of each transcription factor familys members. (FIG. 3b) Up- or down-regulated transcripts from every transcription factor family involved in transcription.

FIG. 4 indicates qRT-PCR expression analysis of 7 transcription factor and 7 heat shock proteins in response to heat stress. qRT-PCR was performed to validate the results of the RNA sequencing analysis using cDNAs prepared from 3-year-old needles of Korean fir exposed for 21 days to control (22° C.) or heat-treatment (30° C.) conditions. Error bars denote standard errors of technical replicates. Expression values of each gene are normalised against the expression of Actin (Uddenberg et al. 2013).

### PREFERRED EMBODIMENT OF INVENTION

Korean fir (Abies koreana) is an endemic and rare species in South Korea, which is sensitive to climate change. In the present invention, the inventors have used next-generation massively parallel sequencing technology and de novo transcriptome assembly to gain a comprehensive overview of the Korean fir transcriptome under heat stress.

The inventors have sequenced control and heat-treated samples of Korean fir, obtaining 183,094,162 and 161,685,060 clean reads, respectively. After de novo assembly and quantitative assessment, 406,207 transcripts were generated with an average length of 532 bp.

Among 8,330 differentially expressed transcripts, 3,721 transcripts being up-regulated and 4,609 transcripts being down-regulated have been detected. A gene ontology analysis of these transcripts reveals to be expressed in response to heat-stress. Further analysis also reveals that 300 transcription factors are differentially expressed. Finally, 14 regulated candidate genes that are associated with heat stress have been examined using quantitative real-time PCR (qRT-PCR).

In the present invention, the inventors have presented the first comprehensive characterization of heat-treated Korean fir using a transcriptome analysis.

The present invention can be explained more concretely as follows.

(1) Transcriptome Sequencing and De Novo Assembly

To elucidate the molecular responses to heat stress in Korean fir, the inventors prepared libraries from heat-treated and control samples for sequencing. In total, 186,191,688 and 164,421,644 raw reads were obtained from the control and heat-treated samples respectively (Table 1). From these samples, 183,094,162 and 161,685,060 clean reads respectively were obtained. Among the clean reads, the Q20 percentage (sequencing error rate <1%) was over 99% and the G+C content was approximately 45% for both libraries (Table 1). Table 1 shows quality of Korean fir's sequencing.

TABLE 1

| | | | | GC | Q20 | Q30 |
|---|---|---|---|---|---|---|
| Sample | Raw reads | Clean reads | Clean bases | (%) | (%) | (%) |
| Control | 186, 191, 688 | 183, 094, 162 | 18, 312, 890, 321 | 44.89 | 99.16 | 96.96 |
| Heat-treated | 164, 421, 644 | 161, 685, 060 | 16, 200, 663, 415 | 45.5 | 99.15 | 96.94 |

Transcriptome de novo assembly was performed using Trinity software, which generated 406,207 transcripts with a mean length of 472.74 bp and an N50 of 532 bp for the merged assembly of both libraries (Table 2). Table 2 shows length distributions of the assembled Avies koreana transcriptions.

TABLE 2

| Type | All transcript contingents |
|---|---|
| Total trinity transcripts | 406,207 |
| Minimum length (bp) | 201 |
| Maximum length (bp) | 19,314 |
| Mean length (bp) | 472.74 |
| N50 (bp) | 532 |
| Total length (bp) | 192,031,706 |

(2) Functional Annotation and Classification of the Korean Fir Transcriptome.

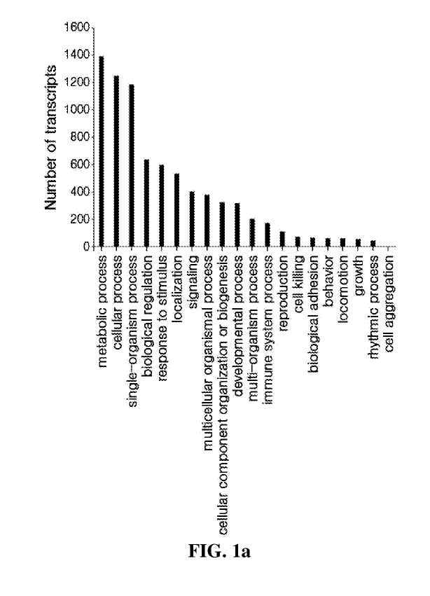

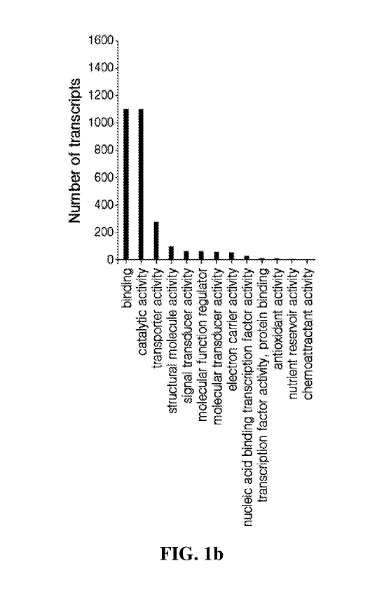

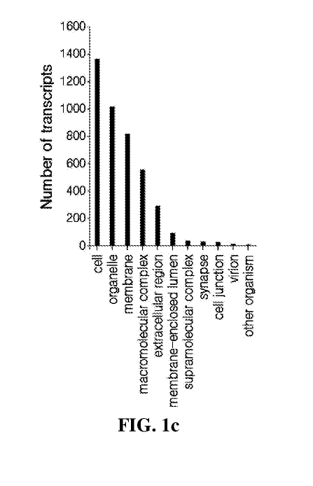

For annotation purposes, the 406,207 assembled transcripts were analysed for gene ontology (GO) terms using Blast2GO. Altogether, 46,603 transcripts, 13.21% of the total assembled transcripts, were annotated using the GO database. The annotated Korean fir transcripts were functionally categorized based on the GO classification system, which contains 3 major functional categories, biological processes, cellular components, and molecular functions (Tables 3-5 and FIG. 1).

For the category of biological process, the most abundant groups were metabolic process (1,392 transcripts), cellular process (1,249 transcripts), single-organism process (1,185 transcripts), biological regulation (636 transcripts), and response to stimulus (598 transcripts). In the molecular function category, composed of 13 functional groups, binding (1,102 transcripts) and catalytic activity (1,100 transcripts) were the most highly represented groups. In cellular component, cell part (1,368 transcripts) and organelle (1,018 transcripts) were the most represented groups.

Table 3 shows GO classification of biological processes functional category.

TABLE 3

| GO-id | GO-term | number of transcript |
|---|---|---|
| GO: 0008152 | metabolic process | 1392 |
| GO: 0009987 | cellular process | 1249 |
| GO: 0044699 | single-organism process | 1185 |
| GO: 0065007 | biological regulation | 636 |
| GO: 0050896 | response to stimulus | 598 |
| GO: 0051179 | localization | 533 |
| GO: 0023052 | signaling | 405 |
| GO: 0032501 | multicellular organismal process | 379 |
| GO: 0071840 | cellular component organization | 325 |
| | or biogenesis | |
| GO: 0032502 | developmental process | 318 |
| GO: 0051704 | multi-organism process | 203 |
| GO: 0002376 | immune system process | 173 |
| GO: 0000003 | reproduction | 111 |
| GO: 0001906 | cell killing | 73 |
| GO: 0022610 | biological adhesion | 66 |
| GO: 0007610 | behavior | 62 |
| GO: 0040011 | locomotion | 60 |
| GO: 0040007 | growth | 55 |
| GO: 0048511 | rhythmic process | 45 |
| GO: 0098743 | cell aggregation | 2 |

Table 4 shows GO classification of molecular functions functional category.

TABLE 4

| | | number of |
|---|---|---|
| GO-id | GO-term | transcript |
| GO: 0005488 | binding | 1102 |
| GO: 0003824 | catalytic activity | 1100 |
| GO: 0005215 | transporter activity | 277 |
| GO: 0005198 | structural molecule activity | 98 |
| GO: 0004871 | signal transducer activity | 63 |
| GO: 0098772 | molecular function regulator | 62 |
| GO: 0060089 | molecular transducer activity | 57 |
| GO: 0009055 | electron carrier activity | 52 |
| GO: 0001071 | nucleic acid binding transcription factor | 28 |
| | activity | |
| GO: 0000988 | transcription factor activity, protein binding | 12 |
| GO: 0016209 | antioxidant activity | 11 |
| GO: 0045735 | nutrient reservoir activity | 5 |
| GO: 0042056 | chemoattractant activity | 2 |

Table 5 shows GO classification of cellular components functional category.

TABLE 5

| GO-id | GO-term | number of transcript |
|---|---|---|
| GO: 0005623 | cell | 1368 |
| GO: 0043226 | organelle | 1018 |
| GO: 0016020 | membrane | 819 |
| GO: 0032991 | macromolecular complex | 557 |
| GO: 0005576 | extracellular region | 292 |
| GO: 0031974 | membrane-enclosed lumen | 93 |
| GO: 0099080 | supramolecular complex | 37 |
| GO: 0045202 | synapse | 32 |
| GO: 0030054 | cell junction | 28 |
| GO: 0019012 | virion | 15 |
| GO: 0044215 | other organism | 9 |

(3) Differentially Expressed Transcripts (DETs) Involved in the Heat-Stress Responses of Korean Fir

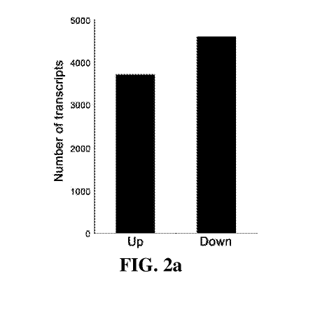

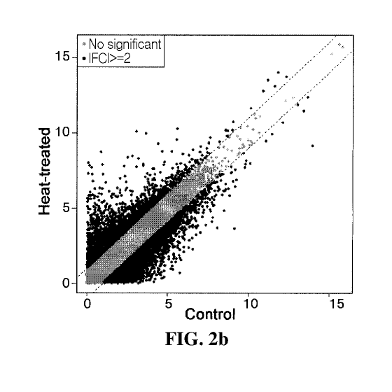

To identify potential heat-stress-responsive genes in Korean fir, the gene expression profiles were compared between control and heat-treated samples. For each transcript of the assembly, the number of mapped reads was compared between the control and the heat-treated samples (FIG. 2). As a result, 8,330 were found to be DETs, with 3,721 up-regulated transcripts and 4,609 down-regulated transcripts in heat-treated sample compared with the control based on the fragments per kb per million fragments method. The distribution of transcript changes is shown in FIG. 2.

The top 20 most enriched functional groups are shown in Table 3. Among these, 11 functional groups (55%), including metabolic process, cellular process, single-organism process, response to stimulus, carbohydrate binding, and heme binding, were significantly enriched within the molecular function category. Four functional groups (20%), including binding, metabolic process, single-organism process, cellular process, were significantly enriched within the molecular functions category, and five functional groups (25%) cell, organelle, membrane, macromolecular complex, and extracellular region, were significantly enriched within the cellular component category. Thus, changes in the biological processes may be very important in response to heat stress in Korean fir.

Table 6 shows top 20 most enriched functional groups in the gene ontology categories.

TABLE 6

| Functional groups | GO-id | Transcript number |
|---|---|---|
| Biological process | | |
| metabolic process | GO: 0008152 | 1191 |
| cellular process | GO: 0009987 | 1008 |
| single-organism process | GO: 0044699 | 992 |
| response to stimulus | GO: 0050896 | 324 |
| cellular component | GO: 0071840 | 174 |
| organization or biogenesis | | |
| developmental process | GO: 0032502 | 149 |
| multi-organism process | GO: 0051704 | 137 |
| Molecular functions | | |
| Binding | GO: 0005488 | 874 |
| metabolic process | GO: 0008152 | 835 |
| single-organism process | GO: 0044699 | 430 |
| cellular process | GO: 0009987 | 277 |
| Cellular components | | |
| Cell | GO: 0005623 | 1119 |
| Organelle | GO: 0043226 | 811 |
| membrane | GO: 0016020 | 638 |
| macromolecular complex | GO: 0032991 | 443 |
| extracellular region | GO: 0005576 | 182 |

To investigate the biological roles of genes regulated by heat stress in Korean fir, the inventors identified DETs (fold change >2) among the enriched GO terms, which were separated into the three main categories, biological processes, molecular functions, and cellular components (Tables 7-9).

Table 7 shows GO annotation of biological processes functional category.

TABLE 7

| GO-id | GO-term | number of transcript |
|---|---|---|
| GO: 0000003 | reproduction | 22 |
| GO: 0001906 | cell killing | 8 |
| GO: 0002376 | immune system process | 67 |
| GO: 0005488 | binding | 8 |
| GO: 0007610 | behavior | 15 |
| GO: 0008152 | metabolic process | 1191 |
| GO: 0009987 | cellular process | 1008 |
| GO: 0022414 | reproductive process | 47 |
| GO: 0022610 | biological adhesion | 38 |
| GO: 0023052 | signaling | 177 |
| GO: 0032501 | multicellular organismal process | 181 |
| GO: 0032502 | developmental process | 149 |
| GO: 0040007 | growth | 24 |
| GO: 0040011 | locomotion | 38 |
| GO: 0044699 | single-organism process | 992 |
| GO: 0048511 | rhythmic process | 4 |
| GO: 0050896 | response to stimulus | 324 |
| GO: 0051179 | localization | 302 |
| GO: 0051704 | multi-organism process | 137 |
| GO: 0060089 | molecular transducer activity | 15 |
| GO: 0065007 | biological regulation | 320 |
| GO: 0071840 | cellular component organization | 174 |
| | or biogenesis | |
| GO: 0098743 | cell aggregation | 2 |

Table 8 shows GO annotation of molecular functions functional category.

TABLE 8

| GO-id | GO-term | number of transcript |
|---|---|---|
| GO: 0002376 | immune system process | 2 |
| GO: 0005198 | structural molecule activity | 68 |
| GO: 0005488 | binding | 874 |
| GO: 0008152 | metabolic process | 835 |
| GO: 0009055 | electron carrier activity | 79 |
| GO: 0009987 | cellular process | 277 |
| GO: 0016209 | antioxidant activity | 9 |
| GO: 0023052 | signaling | 37 |
| GO: 0044699 | single-organism process | 430 |
| GO: 0045735 | nutrient reservoir activity | 6 |
| GO: 0050896 | response to stimulus | 41 |
| GO: 0051179 | localization | 130 |
| GO: 0060089 | molecular transducer activity | 36 |
| GO: 0065007 | biological regulation | 91 |
| GO: 0098772 | molecular function regulator | 42 |

Table 9 shows GO annotation of cellular components functional category.

TABLE 9

| GO-id | GO-term | number of transcript |
|---|---|---|
| GO: 0005576 | extracellular region | 182 |
| GO: 0005623 | cell | 1119 |
| GO: 0016020 | membrane | 638 |
| GO: 0019012 | virion | 32 |
| GO: 0030054 | cell junction | 15 |
| GO: 0031012 | extracellular matrix | 15 |
| GO: 0031974 | membrane-enclosed lumen | 71 |
| GO: 0032991 | macromolecular complex | 443 |
| GO: 0043226 | organelle | 811 |
| GO: 0045202 | synapse | 21 |

(4) Identification of Transcription Factors (TFs) Involved in Heat Stress

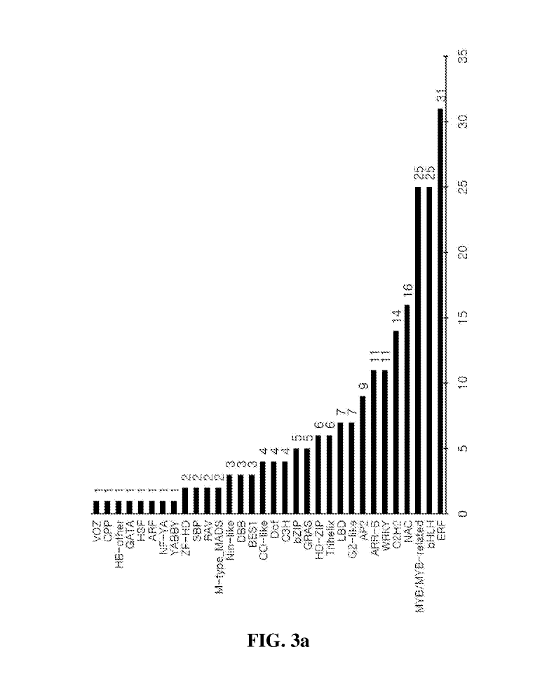

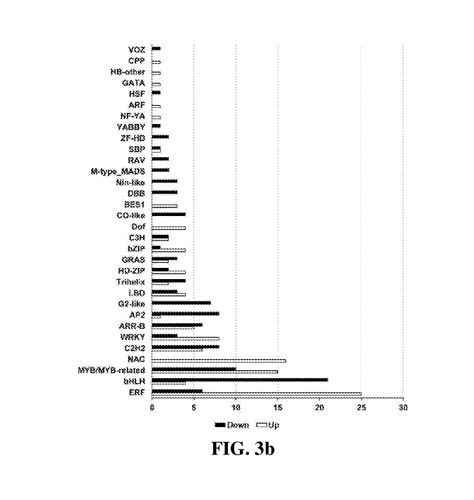

TFs are sequence-specific DNA-binding proteins that interact with the promoter regions of target genes and modulate gene expression. The transcriptional regulation of heat stress has been widely documented in model plants. To identify the TFs involved in heat-stress responses, the inventors surveyed the putative TFs that were differentially expressed in Korean fir under heat stress. The TFs in this study were compared with P. abies transcriptome sequences obtained from publicly available datasets (E-value <1e-10). A total of 8,330 DETs were identified as being involved in transcription, including 215 DETs (111 up-regulated and 104 down-regulated) (Tables 10-19 and FIG. 3).

The largest gene family was the ethylene-responsive element-binding factor family (ERF), followed by the basic helix-loop-helix family (bHLH), MYB/MYB-related, NAC, C2H2 family, and the WRKY family. Of these TF families, ERF, including 31 transcripts (25 up- and 6 down-regulated), bHLH, including 25 transcripts (4 up- and 21 down-regulated), and MYB/MYB-related, including 25 transcripts (15 up- and 10 down-regulated), were the three most enriched TF families. All 16 of the NAC TF family transcripts were up-regulated under heat-treated conditions (FIG. 3). This analysis provided a deeper understanding of the roles of TFs under heat stress.

TABLE 10

| Transcript | Fold change | Accession | Description |
|---|---|---|---|
| c1031_g1_i1 | −7.868031 | MA_112273g0010 | YABBY family protein |
| c173884_g1_i1 | 7.060228 | MA_76955g0010 | bHLH family protein |
| c173884_g1_i2 | 3.963811 | MA_76955g0010 | bHLH family protein |
| c174808_g1_i1 | 2.007338 | MA_8343g0010 | BES1 family protein |
| c175108_g1_i1 | 8.529715 | MA_109421g0010 | C2H2 family protein |
| c175108_g2_i1 | 2.360671 | MA_109421g0010 | C2H2 family protein |
| c59151_g1_i1 | 2.7534 | MA_10431706g0010 | ERF family protein |
| c175993_g1_i2 | −2.28213 | MA_96029g0010 | GRAS family protein |
| c176847_g1_i1 | −2.726416 | MA_21538g0020 | G2-like family protein |
| c176847_g2_i1 | −4.185472 | MA_21538g0020 | G2-like family protein |
| c176974_g1_i1 | −2.110557 | MA_448849g0010 | bHLH family protein |
| c177088_g1_i1 | −2.699163 | MA_333471g0010 | M-type_MADS family |
| | | | protein |
| c178130_g1_i2 | 2.506035 | MA_67841g0010 | ERF family protein |
| c178188_g1_i1 | 2.580721 | MA_40234g0010 | MYB_related family |
| | | | protein |
| c178363_g1_i2 | −2.062606 | MA_20585g0010 | bHLH family protein |
| c181108_g1_i1 | 2.002653 | MA_10426628g0010 | MYB_related family |
| | | | protein |
| c182004_g1_i1 | −2.981429 | MA_126170g0010 | C3H family protein |
| c182125_g1_i1 | 4.114126 | MA_904750g0010 | ERF family protein |
| c183060_g1_i1 | 3.515343 | MA_18454g0020 | ERF family protein |
| c183504_g1_i1 | −2.048848 | MA_908g0010 | MYB family protein |
| c183504_g1_i3 | −2.039392 | MA_908g0010 | MYB family protein |
| c183504_g1_i4 | −2.284153 | MA_908g0010 | MYB family protein |
| c183933_g1_i1 | −2.063262 | MA_18042g0010 | bHLH family protein |
| c183952_g1_i1 | 3.029494 | MA_123810g0010 | Dof family protein |
| c184757_g1_i1 | 9.468423 | MA_168025g0010 | ERF family protein |

The largest gene family was the ethylene-responsive element-binding factor family (ERF), followed by the basic helix-loop-helix family (bHLH), MYB/MYB-related, NAC, C2H2 family, and the WRKY family. Of these TF families, ERF, including 31 transcripts (25 up- and 6 down-regulated), bHLH, including 25 transcripts (4 up- and 21 down-regulated), and MYB/MYB-related, including 25 transcripts (15 up- and 10 down-regulated), were the three most enriched TF families. All 16 of the NAC TF family transcripts were up-regulated under heat-treated conditions (FIG. 3). This analysis provided a deeper understanding of the roles of TFs under heat stress.

TABLE 11

| Transcript | Fold change | Accession | Description |
|---|---|---|---|
| c72602_g1_i1 | −2.151772 | MA_10430620g0010 | MYB_related family |
| | | | protein |
| c185529_g1_i3 | −2.032438 | MA_29238g0010 | C2H2 family protein |
| c186250_g1_i1 | 3.446577 | MA_18939g0010 | NAC family protein |
| c85122_g1_i1 | −2.61958 | MA_33964g0010 | MYB family protein |
| c186255_g1_i3 | −2.186555 | MA_10433428g0010 | bHLH family protein |
| c86779_g1_i1 | 12.349311 | MA_32651g0010 | ERF family protein |
| c186781_g1_i2 | −2.585259 | MA_10437259g0030 | Trihelix family protein |
| c187267_g1_i1 | −2.609357 | MA_29186g0010 | bHLH family protein |
| c187267_g1_i3 | −3.132448 | MA_29186g0010 | bHLH family protein |
| c187596_g1_i1 | 2.591386 | MA_91369g0010 | LBD family protein |
| c187666_g1_i2 | 2.033648 | MA_103616g0010 | WRKY family protein |
| c187666_g1_i3 | 2.107602 | MA_103616g0010 | WRKY family protein |
| c187960_g2_i1 | −2.105997 | MA_83273g0010 | ARR-B family protein |
| c188259_g1_i1 | −2.352565 | MA_9438016g0010 | MYB_related family |
| | | | protein |
| c188290_g2_i2 | 2.350223 | MA_126273g0010 | WRKY family protein |
| c188611_g1_i1 | −2.049829 | MA_5629699g0010 | ERF family protein |
| c188622_g2_i2 | −2.270185 | MA_79519g0010 | Trihelix family protein |

TABLE 12

| Transcript | Fold change | Accession | Description |
|---|---|---|---|
| c92378_g1_i1 | 4.581882 | MA_323706g0010 | MYB family protein |
| c189122_g1_i1 | −3.069444 | MA_9284799g0010 | M-type_MADS family |
| | | | protein |
| c189217_g1_i1 | 11.480822 | MA_137415g0010 | NAC family protein |
| c189403_g3_i1 | 2.009442 | MA_10435070g0010 | NF-YA family protein |
| c189458_g1_i1 | 7.945465 | MA_28894g0010 | ERF family protein |
| c189458_g1_i2 | 6.553164 | MA_28894g0010 | ERF family protein |
| c189548_g1_i1 | −4.621129 | MA_166248g0010 | ERF family protein |
| c189548_g2_i1 | 3.369481 | MA_184464g0010 | ERF family protein |
| c92821_g1_i1 | 2.313337 | MA_179641g0010 | WRKY family protein |
| c189548_g3_i1 | −6.572188 | MA_8552524g0010 | ERF family protein |
| c189572_g2_i1 | 17.996678 | MA_103386g0010 | NAC family protein |
| c189913_g1_i1 | 2.188542 | MA_83273g0010 | ARR-B family protein |
| c189913_g1_i2 | 2.02224 | MA_83273g0010 | ARR-B family protein |
| c190011_g1_i1 | 3.158316 | MA_2446g0010 | ERF family protein |
| c190059_g2_i1 | −3.430555 | MA_181986g0010 | G2-like family protein |
| c190059_g2_i2 | −3.147294 | MA_181986g0010 | G2-like family protein |
| c190059_g2_i3 | −3.335977 | MA_181986g0010 | G2-like family protein |
| c190267_g1_i1 | 5.434037 | MA_17466g0010 | MYB_related family |
| | | | protein |

TABLE 13

| Transcript | Fold change | Accession | Description |
|---|---|---|---|
| c190473_g1_i1 | −2.120294 | MA_795128g0010 | C2H2 family protein |
| c190677_g1_i1 | 10.952408 | MA_96063g0020 | ERF family protein |
| c190805_g1_i1 | −3.159418 | MA_17689g0010 | bHLH family protein |
| c96987_g1_i1 | −2.636113 | MA_65818g0010 | bHLH family protein |
| c96987_g1_i2 | −2.334228 | MA_65818g0010 | bHLH family protein |
| c191565_g2_i1 | 10.622435 | MA_10431706g0010 | ERF family protein |
| c97423_g1_i1 | 3.590895 | MA_98506g0010 | ARF family protein |
| c191814_g1_i3 | −3.434951 | MA_10192193g0020 | CO-like family protein |
| c191814_g1_i4 | −3.471905 | MA_10192193g0020 | CO-like family protein |
| c191814_g1_i5 | −2.075247 | MA_10192193g0020 | CO-like family protein |
| c192109_g1_i1 | −2.150302 | MA_10433513g0010 | DBB family protein |
| c192109_g1_i4 | −2.359486 | MA_10433513g0010 | DBB family protein |
| c192109_g1_i5 | −2.47005 | MA_10433513g0010 | DBB family protein |
| c192739_g1_i1 | 2.733316 | MA_3040g0010 | BES1 family protein |
| c192739_g1_i2 | 3.333982 | MA_3040g0010 | BES1 family protein |
| c193072_g1_i1 | 2.543182 | MA_10435735g0010 | Dof family protein |
| c193072_g2_i1 | 2.38402 | MA_175298g0010 | Dof family protein |
| c193407_g2_i1 | 3.149716 | MA_10434389g0010 | HD-ZIP family protein |
| c194156_g2_i1 | −2.602381 | MA_328535g0010 | LBD family protein |
| c194164_g1_i1 | 2.110726 | MA_89683g0010 | MYB family protein |
| c194866_g1_i1 | −2.095522 | MA_2193g0020 | AP2 family protein |
| c194866_g1_i2 | −2.129714 | MA_2193g0020 | AP2 family protein |
| c194935_g1_i1 | 6.480714 | MA_81029g0010 | ERF family protein |
| c195085_g2_i1 | −2.279258 | MA_23673g0010 | RAV family protein |
| c195127_g1_i1 | 2.287407 | MA_132680g0010 | bHLH family protein |

TABLE 14

| Transcript | Fold change | Accession | Description |
|---|---|---|---|
| c195632_g1_i1 | 2.092237 | MA_52027g0010 | SBP family protein |
| c195768_g1_i1 | 2.524947 | MA_102199g0010 | MYB_related family |
| | | | protein |
| c195768_g1_i2 | 2.108329 | MA_102199g0010 | MYB_related family |
| | | | protein |
| c195768_g1_i5 | 2.211518 | MA_102199g0010 | MYB_related family |
| | | | protein |
| c195943_g1_i1 | 3.287131 | MA_10274g0010 | ERF family protein |
| c195982_g1_i1 | 2.460861 | MA_103475g0010 | bZIP family protein |
| c196090_g3_i1 | −2.178972 | MA_57501g0010 | C2H2 family protein |
| c196090_g3_i2 | −2.734315 | MA_57501g0010 | C2H2 family protein |
| c196090_g3_i3 | −2.700976 | MA_57501g0010 | C2H2 family protein |
| c196090_g3_i4 | −2.58162 | MA_57501g0010 | C2H2 family protein |
| c196090_g3_i5 | −2.824334 | MA_57501g0010 | C2H2 family protein |
| c196593_g1_i1 | −3.539241 | MA_12053g0010 | HD-ZIP family protein |
| c196716_g1_i1 | −2.430143 | MA_292200g0010 | LBD family protein |
| c196716_g1_i5 | −2.251357 | MA_292200g0010 | LBD family protein |
| c196922_g1_i1 | 4.642157 | MA_18454g0020 | ERF family protein |
| c196969_g1_i1 | −2.716243 | MA_92168g0010 | bHLH family protein |
| c197094_g1_i1 | −2.083876 | MA_46112g0010 | bHLH family protein |
| c197094_g1_i2 | −2.088458 | MA_46112g0010 | bHLH family protein |
| c197401_g1_i1 | 2.503986 | MA_53351g0010 | WRKY family protein |
| c197401_g1_i2 | 2.993105 | MA_53351g0010 | WRKY family protein |

TABLE 15

| Transcript | Fold change | Accession | Description |
|---|---|---|---|
| c197663_g1_i1 | 13.051203 | MA_5115g0010 | NAC family protein |
| c197820_g1_i1 | 2.150557 | MA_3313g0010 | Trihelix family protein |
| c197820_g2_i1 | 2.099778 | MA_11552g0010 | Trihelix family protein |
| c198046_g1_i1 | 4.932492 | MA_276627g0010 | LBD family protein |
| c198479_g1_i1 | 2.365983 | MA_8552524g0010 | ERF family protein |
| c198701_g1_i1 | −2.287994 | MA_10431176g0010 | bHLH family protein |
| c198701_g1_i3 | −2.206094 | MA_10431176g0010 | bHLH family protein |
| c198799_g1_i3 | −2.000415 | MA_59421g0010 | bZIP family protein |
| c116916_g1_i1 | −2.338575 | MA_9434330g0010 | HSF family protein |
| c199133_g1_i1 | 2.254209 | MA_161258g0010 | GATA family protein |
| c199182_g1_i1 | −3.035092 | MA_42080g0010 | bHLH family protein |
| c199182_g1_i2 | −4.256182 | MA_42080g0010 | bHLH family protein |
| c199495_g4_i1 | −2.577454 | MA_2026g0010 | MYB family protein |
| c199862_g1_i1 | −2.077189 | MA_92489g0010 | SBP family protein |
| c118781_g1_i1 | 2.408786 | MA_16778g0010 | ERF family protein |

TABLE 16

| Transcript | Fold change | Accession | Description |
|---|---|---|---|
| c200530_g4_i1 | 3.219469 | MA_8965632g0010 | HB-other family protein |
| c119909_g1_i1 | 12.018187 | MA_55357g0010 | LBD family protein |
| c201032_g1_i3 | 2.159299 | MA_10430340g0010 | NAC family protein |
| c201159_g1_i1 | 5.148192 | MA_8980g0010 | NAC family protein |
| c201426_g1_i1 | 2.6107 | MA_690904g0010 | bHLH family protein |
| c124069_g1_i1 | −2.600235 | MA_23673g0010 | RAV family protein |
| c124199_g1_i1 | 15.795691 | MA_10274g0010 | ERF family protein |
| c202465_g1_i2 | 2.825338 | MA_9241385g0010 | HD-ZIP family protein |
| c202465_g3_i1 | 3.445482 | MA_9241385g0010 | HD-ZIP family protein |
| c202465_g3_i2 | 2.575566 | MA_9241385g0010 | HD-ZIP family protein |
| c202531_g1_i1 | −2.279326 | MA_10432914g0010 | WRKY family protein |
| c202531_g1_i2 | −2.667002 | MA_10432914g0010 | WRKY family protein |
| c202692_g1_i1 | −2.363425 | MA_83118g0010 | ERF family protein |
| c202692_g1_i2 | −3.330908 | MA_2040g0010 | ERF family protein |
| c204092_g1_i1 | −2.056178 | MA_118174g0010 | G2-like family protein |
| c204139_g1_i2 | 2.07314 | MA_104763g0010 | C2H2 family protein |
| c138270_g1_i1 | −2.442165 | MA_541749g0010 | G2-like family protein |
| c204784_g1_i1 | −2.430923 | MA_98656g0010 | C3H family protein |

TABLE 17

| Transcript | Fold change | Accession | Description |
|---|---|---|---|
| c204868_g1_i1 | −2.709843 | MA_57426g0010 | ZF-HD family protein |
| c204876_g1_i1 | −2.278366 | MA_10430713g0010 | Trihelix family protein |
| c204903_g1_i2 | 2.026325 | MA_910870g0010 | C3H family protein |
| c205180_g1_i1 | −2.227251 | MA_10434312g0010 | AP2 family protein |
| c205180_g1_i2 | −2.801487 | MA_10434312g0010 | AP2 family protein |
| c205180_g1_i3 | −2.280421 | MA_10434312g0010 | AP2 family protein |
| c205180_g2_i1 | −3.30229 | MA_75070g0010 | AP2 family protein |
| c205180_g2_i3 | −3.748356 | MA_75070g0010 | AP2 family protein |
| c205214_g1_i1 | 2.584727 | MA_70076g0010 | C2H2 family protein |
| c205214_g1_i2 | 2.637969 | MA_70076g0010 | C2H2 family protein |
| c206078_g1_i1 | 4.637825 | MA_1201g0010 | MYB family protein |
| c28471_g1_i1 | −4.036742 | MA_42080g0010 | bHLH family protein |
| c206532_g1_i1 | −2.467213 | MA_10436384g0010 | Nin-like family protein |
| c206532_g1_i2 | −2.613473 | MA_10436384g0010 | Nin-like family protein |
| c206532_g1_i3 | −2.544505 | MA_10436384g0010 | Nin-like family protein |
| c141864_g1_i1 | 3.245759 | MA_101790g0010 | MYB family protein |
| c142609_g1_i1 | 84.6609 | MA_75192g0010 | NAC family protein |
| c207008_g1_i1 | −2.523375 | MA_10433418g0010 | bHLH family protein |
| c207159_g1_i1 | 40.422026 | MA_37058g0010 | MYB family protein |
| c207251_g1_i1 | 4.833634 | MA_35014g0010 | bZIP family protein |
| c207251_g1_i2 | 5.401583 | MA_35014g0010 | bZIP family protein |

TABLE 18

| Transcript | Fold change | Accession | Description |
|---|---|---|---|
| c207449_g1_i1 | 3.487596 | MA_10031781g0010 | ERF family protein |
| c207449_g1_i3 | 2.571808 | MA_10031781g0010 | ERF family protein |
| c207449_g1_i5 | 2.329291 | MA_10031781g0010 | ERF family protein |
| c207684_g1_i1 | −2.266166 | MA_130948g0020 | AP2 family protein |
| c145812_g1_i1 | 4.681697 | MA_4032g0010 | ERF family protein |
| c208452_g1_i1 | −2.243217 | MA_92689g0020 | ARR-B family protein |
| c208900_g2_i1 | 7.946934 | MA_79692g0010 | LBD family protein |
| c209398_g1_i1 | 2.117936 | MA_10426586g0010 | ERF family protein |
| c149489_g1_i1 | −2.987342 | MA_10192193g0020 | CO-like family protein |
| c210811_g1_i1 | 4.325053 | MA_121533g0010 | MYB family protein |
| c149930_g1_i1 | −2.722994 | MA_36755g0010 | ZF-HD family protein |
| c150425_g1_i1 | −2.345047 | MA_908g0010 | MYB family protein |
| c211518_g1_i2 | 2.114335 | MA_44659g0010 | CPP family protein |
| c151473_g1_i1 | 11.570553 | MA_10048467g0010 | MYB family protein |
| c211987_g3_i1 | 3.013581 | MA_10435790g0010 | GRAS family protein |
| c211987_g3_i3 | 2.586252 | MA_10435790g0010 | GRAS family protein |
| c211987_g4_i2 | −2.077267 | MA_10435790g0010 | GRAS family protein |
| c212616_g3_i1 | −2.317569 | MA_81876g0010 | C2H2 family protein |
| c213140_g1_i1 | 2.210154 | MA_88541g0010 | C3H family protein |
| c213518_g1_i1 | 2.101457 | MA_10432457g0010 | ARR-B family protein |
| c213518_g1_i3 | 2.010442 | MA_10432457g0010 | ARR-B family protein |

TABLE 19

| Transcript | Fold change | Accession | Description |
|---|---|---|---|
| c213518_g1_i5 | 2.140258 | MA_10432457g0010 | ARR-B family protein |
| c154949_g1_i1 | 2.27663 | MA_67041g0010 | AP2 family protein |
| c214439_g3_i1 | 2.072778 | MA_98483g0010 | NAC family protein |
| c214439_g2_i3 | 2.515361 | MA_98483g0010 | NAC family protein |
| c214439_g4_i1 | 3.840586 | MA_18153g0010 | NAC family protein |
| c214439_g2_i5 | 4.613868 | MA_98483g0010 | NAC family protein |
| c214439_g2_i6 | 3.141246 | MA_98483g0010 | NAC family protein |
| c214439_g2_i7 | 4.805022 | MA_98483g0010 | NAC family protein |
| c214439_g2_i9 | 2.190435 | MA_98483g0010 | NAC family protein |
| c214465_g4_i3 | −2.362063 | MA_74833g0010 | WRKY family protein |
| c214536_g2_i1 | 2.091411 | MA_41803g0010 | MYB_related family |
| | | | protein |
| c215352_g1_i1 | 2.334521 | MA_70076g0010 | C2H2 family protein |
| c215417_g4_i1 | 2.798689 | MA_86256g0010 | NAC family protein |
| c216203_g5_i2 | −2.270643 | MA_93471g0010 | HD-ZIP family protein |
| c216275_g6_i1 | −2.708492 | MA_130776g0010 | bHLH family protein |
| c216275_g6_i2 | −2.706876 | MA_130776g0010 | bHLH family protein |
| c216369_g1_i1 | −2.184289 | MA_78829g0010 | ARR-B family protein |
| c216369_g1_i2 | −2.200486 | MA_78829g0010 | ARR-B family protein |
| c216369_g1_i3 | −2.067379 | MA_78829g0010 | ARR-B family protein |
| c216369_g1_i6 | −2.405516 | MA_78829g0010 | ARR-B family protein |
| c219427_g1_i1 | 13.574547 | MA_103386g0010 | NAC family protein |
| c163512_g2_i1 | −2.369208 | MA_4766093g0010 | GRAS family protein |
| c277094_g1_i1 | 3.827784 | MA_5979847g0010 | ERF family protein |
| c323696_g1_i1 | −2.238866 | MA_10430713g0010 | Trihelix family protein |
| c37207_g1_i1 | −2.125095 | MA_16454g0010 | bHLH family protein |
| c164712_g1_i1 | 7.328416 | MA_212937g0010 | WRKY family protein |
| c164712_g1_i2 | 6.520219 | MA_212937g0010 | WRKY family protein |
| c165310_g2_i1 | −2.521929 | MA_93127g0010 | MYB family protein |
| c167914_g1_i1 | 2.863024 | MA_208967g0010 | MYB_related family |
| | | | protein |
| c168362_g1_i1 | 7.398173 | MA_502153g0010 | ERF family protein |
| c168621_g1_i1 | −2.629838 | MA_10433310g0010 | ERF family protein |
| c169946_g1_i1 | 3.422148 | MA_944867g0010 | Dof family protein |
| c170791_g1_i1 | −2.120787 | MA_9374017g0010 | MYB family protein |
| c170899_g1_i1 | 2.297978 | MA_33471g0010 | bZIP family protein |
| c172322_g1_i1 | −3.14497 | MA_69872g0010 | VOZ family protein |

(5) Identification of Heat Shock Proteins (Hsps)

To begin to elucidate the molecular basis of heat-stress tolerance in Korean fir, we sought to identify sequences in the transcriptome that encoded Hsps. Based on sequence conservation (E<1-10), the inventors identified 114 putative Hsp transcripts (Tables 20-24). Most of the Hsps were significantly up-regulated during the heat treatment (Table 25). Of these transcripts, Trans Decoder identified 36 complete open reading frames with putative start and stop codons (Tables 26-27). Thus, these transcripts could be used in further analysis (gene functional responses to heat).

Tables 20-24 show the lists of putative heat shock protein (Hsp) transcripts of Korean fir.

TABLE 20
Hsp90

| Transcript | Annotation | Fold change |
|---|---|---|
| c188934_g1_i1 | HSP90_0278\|Vi + C2:C37tis vinifera | 9.346901 |
| c209838_g2_i4 | HSP90_0266\|Vitis vinifera | 3.683746 |
| c217843_g2_i1 | HSP90_0236\|Ricinus communis | 3.045776 |
| c214101_g2_i2 | HSP90_0266\|Vitis vinifera | 3.002485 |
| c212080_g4_i1 | \|HSP90_0281\|Vitis vinifera | 2.994265 |
| c211524_g1_i1 | \|HSP90_0200\|Arabidopsis thaliana | 2.920935 |
| c196916_g1_i1 | HSP90_0266\|Vitis vinifera | 2.883347 |
| c205616_g1_i1 | HSP90_0266\|Vitis vinifera | 2.839909 |
| c217670_g2_i1 | HSP90_0266\|Vitis vinifera | 2.637779 |
| c218714_g1_i1 | HSP90_0266\|Vitis vinifera | 2.630367 |
| c217843_g1_i2 | HSP90_0224\|Physcomitrella patens | 2.571138 |
| | subsp. Patens | |
| c218733_g1_i1 | HSP90_0266\|Vitis vinifera | 2.522455 |
| c212628_g3_i1 | HSP90_0266\|Vitis vinifera | 2.49679 |
| c218745_g3_i2 | HSP90_0266\|Vitis vinifera | 2.432798 |
| c207957_g1_i1 | \|HSP90_0281\|Vitis vinifera | 2.414131 |
| c218389_g1_i1 | HSP90_0266\|Vitis vinifera | 2.360389 |
| c213225_g1_i1 | HSP90_0266\|Vitis vinifera | 2.355805 |
| c218393_g1_i1 | HSP90_0266\|Vitis vinifera | 2.334724 |
| c205368_g1_i1 | HSP90_0266\|Vitis vinifera | 2.318561 |
| c218339_g1_i1 | HSP90_0266\|Vitis vinifera | 2.242278 |
| c218631_g2_i1 | HSP90_0266\|Vitis vinifera | 2.232708 |
| c218392_g1_i1 | HSP90_0266\|Vitis vinifera | 2.230957 |
| c218580_g2_i2 | HSP90_0266\|Vitis vinifera | 2.198331 |
| c217680_g1_i1 | HSP90_0266\|Vitis vinifera | 2.167373 |
| c217948_g1_i1 | HSP90_0266\|Vitis vinifera | 2.098881 |
| c218193_g1_i2 | HSP90_0266\|Vitis vinifera | 2.094373 |
| c218689_g1_i1 | HSP90_0266\|Vitis vinifera | 2.093084 |
| c218683_g3_i1 | HSP90_0266\|Vitis vinifera | 2.057964 |
| c218736_g1_i3 | HSP90_0266\|Vitis vinifera | 2.057394 |
| c218736_g1_i3 | HSP90_0266\|Vitis vinifera | 2.057394 |
| c273438_g1_i1 | HSP90_0266\|Vitis vinifera | 2.051236 |
| c218750_g1_i1 | HSP90_0266\|Vitis vinifera | 2.037203 |
| c189979_g1_i1 | HSP90_0208\|Glycine max | 2.005698 |
| c183916_g1_i1 | HSP90_0266\|Vitis vinifera | −4.707293 |
| c213970_g4_i1 | HSP90_0266\|Vitis vinifera | −3.645057 |
| c205143_g5_i1 | HSP90_0266\|Vitis vinifera | −2.118258 |
| c149565_g1_i1 | HSP70_1146\|Vigna radiata | 8.551416 |

TABLE 21
Hsp70

| Transcript | Annotation | Fold change |
|---|---|---|
| c210065_g3_i1 | HSP70_0966\|Cucumis sativus | 7.988422 |
| c210065_g1_i1 | HSP70_1095\|Solanum lycopersicum | 6.789687 |
| c201565_g1_i1 | HSP70_1078\|Ricinus communis | 6.746024 |
| c201565_g1_i2 | HSP70_1078\|Ricinus communis | 5.987419 |
| c151899_g1_i2 | HSP70_1149\|Vitis vinifera | 4.67817 |
| c207016_g1_i1 | HSP70_0928\|Arabidopsis thaliana | 3.803087 |
| c151899_g1_i1 | HSP70_1149\|Vitis vinifera | 3.5109 |
| c194240_g1_i1 | HSP70_1154\|Vitis vinifera | 3.123973 |
| c203374_g1_i1 | HSP70_1077\|Ricinus communis | 2.764551 |
| c188327_g1_i1 | HSP70_1077\|Ricinus communis | 2.56852 |
| c201131_g1_i1 | HSP70_1084\|Ricinus communis | 2.30653 |
| c136617_g1_i1 | HSP70_0968\|Glycine max | 2.133849 |
| c149639_g1_i1 | HSP70_1102\|Sorghum bicolor | −5.311019 |
| c202543_g1_i1 | HSP70_1149\|Vitis vinifera | −4.61517 |
| c216401_g1_i1 | HSP70_1059\|Physcomitrella patens | −4.032254 |
| | subsp. patens | |
| c213928_g1_i1 | HSP70_1048\|Physcomitrella patens | −3.05916 |
| | subsp. patens | |
| c213499_g1_i1 | HSP70_1083\|Ricinus communis | −2.183558 |

TABLE 22
Hsp60

| Transcript | Annotation | Fold change |
|---|---|---|
| c199303_g3_i1 | HSP60_1249\|Vitis vinifera PN40024 | 15.010842 |
| c59361_g1_i1 | HSP60_1249\|Vitis vinifera PN40024 | 3.223259 |
| c181309_g1_i1 | HSP60_1249\|Vitis vinifera PN40024 | 3.076203 |
| c213126_g3_i1 | HSP60_1249\|Vitis vinifera PN40024 | 2.777554 |
| c212961_g6_i1 | HSP60_1249\|Vitis vinifera PN40024 | 2.614552 |
| c172354_g1_i1 | HSP60_1249\|Vitis vinifera PN40024 | 2.449014 |
| c205069_g6_i1 | HSP60_1249\|Vitis vinifera PN40024 | 2.421496 |
| c202756_g3_i1 | HSP60_1249\|Vitis vinifera PN40024 | 2.407392 |
| c206995_g8_i1 | HSP60_1249\|Vitis vinifera PN40024 | 2.389699 |
| c205375_g11_i1 | HSP60_1249\|Vitis vinifera PN40024 | 2.318159 |
| c205375_g10_i2 | HSP60_1249\|Vitis vinifera PN40024 | 2.307531 |
| c199173_g12_i1 | HSP60_1249\|Vitis vinifera PN40024 | 2.279072 |
| c217918_g4_i2 | HSP60_1249\|Vitis vinifera PN40024 | 2.222044 |
| c210652_g7_i1 | HSP60_1249\|Vitis vinifera PN40024 | 2.214315 |
| c208939_g1_i1 | HSP60_1249\|Vitis vinifera PN40024 | 2.183807 |
| c146029_g1_i1 | HSP60_1249\|Vitis vinifera PN40024 | 2.182885 |
| c202538_g5_i1 | HSP60_1249\|Vitis vinifera PN40024 | 2.181807 |
| c197287_g2_i1 | HSP60_1155\|Ricinus communis | 2.181776 |
| c208500_g6_i1 | HSP60_1249\|Vitis vinifera PN40024 | 2.17939 |
| c65680_g1_i1 | HSP60_1249\|Vitis vinifera PN40024 | 2.168742 |
| c89465_g1_i1 | HSP60_1249\|Vitis vinifera PN40024 | 2.152475 |
| c206337_g6_i1 | HSP60_1249\|Vitis vinifera PN40024 | 2.151082 |
| c72653_g1_i1 | HSP60_1249\|Vitis vinifera PN40024 | 2.142406 |
| c209821_g7_i1 | HSP60_1249\|Vitis vinifera PN40024 | 2.13706 |

TABLE 23

| | | |
|---|---|---|
| c176519_g3_i1 | HSP60_1249\|Vitis vinifera PN40024 | 2.134761 |
| c211942_g2_i1 | HSP60_1249\|Vitis vinifera PN40024 | 2.116001 |
| c175194_g2_i1 | HSP60_1249\|Vitis vinifera PN40024 | 2.082893 |
| c201085_g7_i1 | HSP60_1249\|Vitis vinifera PN40024 | 2.077141 |
| c207663_g5_i1 | HSP60_1249\|Vitis vinifera PN40024 | 2.071007 |
| c209174_g9_i1 | HSP60_1249\|Vitis vinifera PN40024 | 2.053168 |
| c208546_g10_i1 | HSP60_1249\|Vitis vinifera PN40024 | 2.046571 |
| c204736_g10_i1 | HSP60_1249\|Vitis vinifera PN40024 | 2.033637 |
| c182081_g1_i1 | HSP60_1249\|Vitis vinifera PN40024 | 2.024415 |
| c212895_g1_i1 | HSP60_1249\|Vitis vinifera PN40024 | 2.024166 |
| c208546_g5_i1 | HSP60_1249\|Vitis vinifera PN40024 | 2.017233 |
| c323326_g1_i1 | HSP60_1249\|Vitis vinifera PN40024 | 2.013633 |
| c210441_g18_i1 | HSP60_1249\|Vitis vinifera PN40024 | 2.00684 |
| c179788_g2_i1 | HSP60_1249\|Vitis vinifera PN40024 | 2.004798 |
| c209484_g7_i1 | HSP60_1249\|Vitis vinifera PN40024 | 2.004601 |
| c205321_g1_i3 | HSP60_1249\|Vitis vinifera PN40024 | −2.947549 |
| c205932_g9_i1 | HSP60_1249\|Vitis vinifera PN40024 | −2.691181 |
| c196951_g7_i1 | HSP60_1249\|Vitis vinifera PN40024 | −2.491317 |
| c202538_g8_i1 | HSP60_1249\|Vitis vinifera PN40024 | −2.455062 |
| c352458_g1_i1 | HSP60_1249\|Vitis vinifera PN40024 | −2.385704 |
| c273072_g1_i1 | HSP60_1249\|Vitis vinifera PN40024 | −2.364641 |
| c217918_g11_i1 | HSP60_1249\|Vitis vinifera PN40024 | −2.305914 |
| c165229_g1_i1 | HSP60_1249\|Vitis vinifera PN40024 | −2.127454 |
| c128348_g1_i1 | HSP60_1249\|Vitis vinifera PN40024 | −2.031496 |

TABLE 24
shsp

| Transcript | Annotation | Fold change |
|---|---|---|
| c156586_g1_i1 | sHsp_0687\|Ricinus communis | 13.920515 |
| c213666_g1_i5 | sHsp_0673\|Ricinus communis | 11.35502 |
| c201988_g1_i1 | sHsp_0819\|Vitis vinifera PN40024 | 7.295365 |
| c207145_g1_i1 | sHsp_0659\|Physcomitrella patens | 6.147673 |
| | subsp. patens | |
| c213666_g1_i4 | sHsp_0673\|Ricinus communis | 6.134323 |
| c203504_g1_i1 | sHsp_0862\|Vitis vinifera PN40024 | 3.702819 |
| c203504_g1_i2 | sHsp_0862\|Vitis vinifera PN40024 | 3.641076 |
| c213666_g1_i3 | sHsp_0673\|Ricinus communis | 3.155806 |
| c213666_g1_i6 | Hsp_0671\|Ricinus communis | 2.411653 |
| c204834_g1_i1 | sHsp_0673\|Ricinus communis | 2.163703 |
| c200955_g1_i1 | sHsp_0824\|Vitis vinifera PN40024 | 2.133822 |
| c167318_g2_i1 | Hsp_0572\|Nicotiana tabacum | 2.039078 |
| c208399_g1_i3 | sHsp_0600\|Oryza sativa Indica group | −3.738818 |

Table 25 shows differentially expressed Abies koreana transcripts identified as heat shock protein (Hsp) families

TABLE 25

| | | | Fold |
|---|---|---|---|
| Classification | Contigs | Annotation | change |
| Hsp90 | c188934_g1_i1 | HSP90_0278\|Vi + C2:C37tis vinifera | 9.35 |
| | c217843_g2_i1 | HSP90_0236\|Ricinus communis | 3.05 |
| | c212080_g4_i1 | HSP90_0281\|Vitis vinifera | 2.99 |
| | c211524_g1_i1 | HSP90_0200\|Arabidopsis thaliana | 2.92 |
| | c207957_g1_i1 | HSP90_0281\|Vitis vinifera | 2.41 |
| | c218389_g1_i1 | HSP90_0266\|Vitis vinifera | 2.36 |
| | c218689_g1_i1 | HSP90_0266\|Vitis vinifera | 2.09 |
| | c273438_g1_i1 | HSP90_0266\|Vitis vinifera | 2.05 |
| | c218750_g1_i1 | HSP90_0266\|Vitis vinifera | 2.04 |
| | c189979_g1_i1 | HSP90_0208\|Glycine max | 2.01 |
| | c205143_g5_i1 | HSP90_0266\|Vitis vinifera | −2.12 |
| Hsp70 | c149565_g1_i1 | HSP70_1146\|Vigna radiata | 8.55 |
| | c210065_g1_i1 | HSP70_1095\|Solanum lycopersicum | 6.79 |
| | c201565_g1_i1 | HSP70_1078\|Ricinus communis | 6.75 |
| | c201565_g1_i2 | HSP70_1078\|Ricinus communis | 5.99 |
| | c151899_g1_i2 | HSP70_1149\|Vitis vinifera | 4.68 |
| | c207016_g1_i1 | HSP70_0928\|Arabidopsis thaliana | 3.80 |
| | c151899_g1_i1 | HSP70_1149\|Vitis vinifera | 3.51 |
| | c194240_g1_i1 | HSP70_1154\|Vitis vinifera | 3.12 |
| | c203374_g1_i1 | HSP70_1077\|Ricinus communis | 2.76 |
| | c188327_g1_i1 | HSP70_1077\|Ricinus communis | 2.57 |
| | c149639_g1_i1 | HSP70_1102\|Sorghum bicolor | −5.31 |
| | c202543_g1_i1 | HSP70_1149\|Vitis vinifera | −4.62 |
| Hsp60 | c199303_g3_i1 | HSP60_1249\|Vitis vinifera PN40024 | 15.01 |
| | c197287_g2_i1 | HSP60_1155\|Ricinus communis | 2.18 |
| | c212895_g1_i1 | HSP60_1249\|Vitis vinifera PN40024 | 2.02 |
| sHsp | c156586_g1_i1 | sHsp_0687\|Ricinus communis | 13.92 |
| | c213666_g1_i5 | sHsp_0673\|Ricinus communis | 11.36 |
| | c201988_g1_i1 | sHsp_0819\|Vitis vinifera PN40024 | 7.30 |
| | c207145_g1_i1 | sHsp_0659\|Physcomitrella patens subsp. patens | 6.15 |
| | c213666_g1_i4 | sHsp_0673\|Ricinus communis | 6.13 |
| | c203504_g1_i1 | sHsp_0862\|Vitis vinifera PN40024 | 3.70 |
| | c203504_g1_i2 | sHsp_0862\|Vitis vinifera PN40024 | 3.64 |
| | c213666_g1_i3 | sHsp_0673\|Ricinus communis | 3.16 |
| | c213666_g1_i6 | Hsp_0671\|Ricinus communis | 2.41 |
| | c204834_g1_i1 | sHsp_0673\|Ricinus communis | 2.16 |
| Average | | | 4.08 |
| expression | | | |
| levels | | | |

(6) Validation of DETs Using qRT-PCR

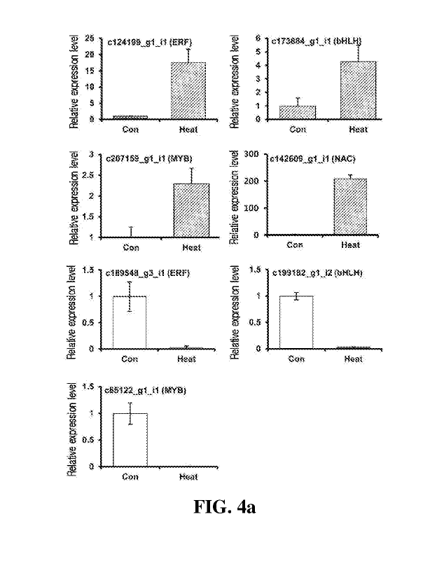

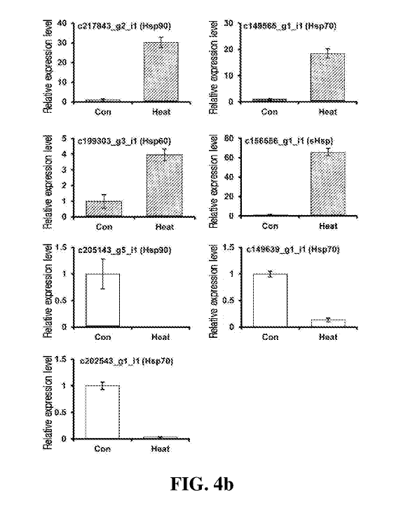

To confirm the accuracy of the RNA-seq results, 14 DETs, including TFs and putative Hsp transcripts, were selected for a qRT-PCR-based comparison of their expression levels between the control and heat-treated samples (FIG. 4). The primer sequences are listed in Tables 26-27. All 14 DETs in the control and heat-treated samples showed the same expression patterns in the qRT-PCR (FIG. 4).

The transcripts included seven putative heat-related TFs. The heat treatment up-regulated c124199_g1_i1 (ERF), c173884_g1_i1 (bHLH), c207159_g1_i1 (MYB), and c142609_g1_i1 (NAC) and down-regulated c189548_g3_i1 (ERF), c199182_g1_i2 (bHLH), and c85122_g1_i1 (MYB) (FIG. 4a). The remaining seven transcripts encoded Hsps. The expression levels of c217843_g2_i1 (Hsp90), c149565_g1_i1 (Hsp70), c199303_g3_i1 (Hsp60), and c156586_g1_i1 (sHsp) were up-regulated by heat-treatment (FIG. 4b), while the expression levels of c205143_g5_i1 (Hsp90), c149639_g1_i1 (Hsp70), and c202543_g1_i1 (Hsp70) were down-regulated by heat-treatment (FIG. 4b). This independent evaluation confirmed the reliability of the RNA-seq data and that these 14 transcript were involved in responses to heat.

Tables 26-27 show primer sequences used for qRT-PCR.

TABLE 26

| | | | | Expected |
|---|---|---|---|---|
| Transcript | Description | Forward primers | Reverse primers | size (bp) |
| c142609_g1_i1 | NAC family | 5′-TGGCTGCAGAGCTCCTTTGA | 5′-TCTGGAGCACACAACCAGCA | 174 |
| (SEQ ID NO: 1) | protein | (SEQ ID NO: 15) | (SEQ ID NO: 16) | |
| c207159_f1_i1 | MYB family | 5′-AGGATGGTCGGCCTGTGTCT | 5′-CAACCCCCGCGATTGAGACC | 200 |
| (SEQ ID NO: 2) | protein | (SEQ ID NO: 17) | (SEQ ID NO: 18) | |
| c124199_g1_i1 | ERF family | 5′-TCGCCGCCATTACCGACTTC | 5′-ATTGCGGGGATGGGTTCTCG | 177 |
| (SEQ ID NO: 3) | protein | (SEQ ID NO: 19) | (SEQ ID NO: 20) | |
| c173884_g1_i1 | bHLH family | 5′-CGCCGAGCGTAACAGGAGAG | 5′-TCGAGCTCATCCACTTGGCG | 150 |
| (SEQ ID NO: 4) | protein | (SEQ ID NO: 21) | (SEQ ID NO: 22) | |
| c85122_g1_i1 | MYB family | 5′-CCAACGCGGCAACTGCTAAT | 5′-ATCCCGCGTCGAATGCTGAT | 114 |
| (SEQ ID NO: 5) | protein | (SEQ ID NO: 23) | (SEQ ID NO: 24) | |
| c199182_g1_i2 | bHLH family | 5′-AGCGGTCTGTTCCGACGATT | 5′-CCGCCATGACCGTCGATTTC | 113 |
| (SEQ ID NO: 6) | protein | (SEQ ID NO: 25) | (SEQ ID NO: 26) | |
| c189548_g3_i1 | ERF family | 5′-CCGCCGAAGAAACCGATGAC | 5′-AAGGTGCCGAGCCAAACTCT | 131 |
| (SEQ ID NO: 7) | protein | (SEQ ID NO: 27) | (SEQ ID NO: 28) | |

TABLE 27

| | | | | Expected |
|---|---|---|---|---|
| Transcript | Description | Forward primers | Reverse primers | size (bp) |
| c217843_g2_i1 | HSP90_0278\| | 5′-ACGTCAGTCCTCCCAA | 5′-CATTGGCCCGCAGTGA | 123 |
| (SEQ ID NO: 8) | V + C2: | GGTG | CTTG | |
| | tisvinifera | (SEQ ID NO: 29) | (SEQ ID NO: 30) | |
| c149565_g1_i1 | HSP70_1146\| | 5′-TGTCCAAGCCGCCATT | 5′-TCATTACGCCTCCCGC | 110 |
| (SEQ ID NO: 9) | Vigna | CTGA | AGTT | |
| | radiata | (SEQ ID NO: 31) | (SEQ ID NO: 32) | |
| c199303_g3_i1 | HSP60_1249\| | 5′-CCGTTGGTGCCCAATT | 5′-CAAATCGTGCAGCACA | 197 |
| (SEQ ID NO: 10) | Vitis | CGAG | GGCA | |
| | vinifera | (SEQ ID NO: 33) | (SEQ ID NO: 34) | |
| | PN40024 | | | |
| c156586_g1_i1 | sHsp_0687\| | 5′-AGCAGCTGAATCCGGA | 5′-CTTAGGTTTCTCGGCCT | 176 |
| (SEQ ID NO: 11) | Ricinus | GGTG | CGGA | |
| | communis | (SEQ ID NO: 35) | (SEQ ID NO: 36) | |
| c205143_g5_i1 | HSP90_0266\| | 5′-AGAGCCAAGCTCCACA | 5′-GAGGGCACCCTTGCGTT | 114 |
| (SEQ ID NO: 12) | Vitis | GGGA | TCT | |
| | vinifera | (SEQ ID NO: 37) | (SEQ ID NO: 38) | |
| c149639_g1_i1 | HSP70_1102\| | 5′-AGCTGCGTAGCTGTAT | 5′-TACGGGATTCATGGCGG | 147 |
| (SEQ ID NO: 13) | Sorghum | GGCA | CTT | |
| | bicolor | (SEQ ID NO: 39) | (SEQ ID NO: 40) | |
| c202543_g1_i1 | HSP70_1149\| | 5′-GGCTCCTTCCGACGAG | 5′-GGCCTCTGCCGATCTCA | 158 |
| (SEQ ID NO: 14) | Vitis | GTAG | AGT | |
| | vinifera | (SEQ ID NO: 41) | (SEQ ID NO: 42) | |
| Actin | Peaccea | 5′-ATTGGGATGGAAGCTG | 5′-CCCACCACTAAGCACAA | |
| | Abies | CTG | TG | |
| | actin | | | |

In the absence of a whole genome sequence, RNA-seq is very successful application tool for comprehensive studies of gene expression and the detection of novel transcripts associated with valuable traits. In this invention, the inventors implemented a de novo RNA-seq technology to obtain insights into the transcriptomic responses induced by heat stress in Korean fir.

A whole-transcriptome analysis was performed in both heat-stressed and unstressed plants. For each sample, more than 160 M high-quality clean reads were obtained, which were de novo assembled into 406,207 transcripts with an N50 of 530 bp (Table 1 and Table 2), which indicates a high quality assembly that includes many full-length cDNAs.

Functional annotation and classification provide predicted information on inner-cell metabolic pathways and the biological behaviors of genes. GO is an internationally standardized gene functional classification system that offers a dynamic-updated controlled vocabulary and a strictly defined structure to describe the properties of genes and their products in any organism.

Among the transcripts, 46,603 (13.21%) known proteins were assigned to GO classes. However, a large proportion of transcripts (86.79%) failed to match these databases owing to the paucity of gene information for Abies. According to the GO classification, cellular process, cell part, and cell were largest groups in the three main GO categories of biological processes, cellular components, and molecular functions, respectively (FIG. 1). Our GO classifications of the annotated transcripts provide a general gene expression profile signature for Korean fir (A. koreana) that will facilitate further studies in Abies.

We than analysed the transcripts that were differentially expressed in the heat-treated and control samples. Under heat stress, the GO category of biological processes (Table 3) was enriched. The largest proportion of the terms were included the metabolic process, cellular process, and single-organism process, indicating that comprehensive changes in Korean fir gene expression levels occurred after the heat treatment. These findings indicated that biological process is significantly changed by responses to heat stress. Additionally, many transcripts were over-represented as belonging to response to stimulus in the heat-treated sample and these transcripts represented the most important components directly involved in protecting plants from stress.

TFs are sequence-specific DNA-binding proteins that interact with cis-elements in the promoter regions of target genes and modulate gene expression. These TFs regulate gene transcription in response to biotic and abiotic stresses, such as cold, high temperatures, high salinity, drought, and pathogen attacks. As the results, several TF families were identified as being involved in heat-stress responses, including ERF, bHLH, MYB/MYB-related, NAC, C2H2 and WRKY (FIG. 3).

The greatest number of ERF family genes are heat-response TFs, and an ERF coactivator gene is synergistically expressed with ERFs under heat stress. The expressions of AtERF53 and ERF1 are induced by heat treatment in Arabidopsis and pakchoi, respectively. The DREB2s TF group belongs to the AP2/ERF family, and it has been characterized in the heat regulatory pathway.

The induced DREB2 functions to enhance heat tolerance in various plants. Other TFs, including bHLH, MYB, and C2H2 families, were also up-regulated during heat treatments and members of these families function in heat tolerance. The ERF, bHLH, MYB, and C2H2 pathways are conserved in Korean firs responses to heat stress. The plant-specific NAC TF family has been implicated in the regulation of diverse processes, including hormone signalling, defence, and stress tolerance. NAC TFs in plants are mainly involved in osmotic stresses, including drought and high salinity.

However, some NACs (RD26) function in response to cold stress. Morishita et al. also reported that ANAC078 in the NAC group TIP is responsive to a combination of high light and heat stress. The inventors found 16 transcripts encoding NAC TF domains, and all of the transcripts were up-regulated and showed significant expression levels by RNA-seq and qRT-PCR (Tables 10-19 and FIG. 4). These results may help to explain the more important functions of the NAC family of genes in the heat responses of Korean fir.

The inventors found only one transcriptional heat shock factor (HSF), which was down-regulated in our results. HSF TFs are key regulators involved in responses to heat stress. The reduction in HSFs (FIG. 4) revealed that the heat-response pathway might have different signalling networks in Korean fir. In addition, several novel TF families (ARR-B, AP2, C3H, and G2-like; FIG. 3) were also identified.

Their homologs in other plant species have not yet been reported in response to heat stress, suggesting that these genes might be specific to Abies species and are attractive targets for further functional characterization. These findings facilitate potential studies focusing on the interactions of different TFs in the regulation of heat stress. Thus, there are considerable conserved and varied components involved in heat-stress response mechanisms across plant species.

The analyses of transcriptome profiles in plants after heat treatment have indicated that the HSP family plays a central role in heat-stress responses. Hsp families, including Hsp100, Hsp90, Hsp70, Hsp60, and small Hsps, are involve in folding and assembling proteins, maintaining protein stabilization, activating proteins, and degrading proteins in many normal cellular processes and under stress conditions.

In the present invention, the expression levels of most Hsp genes in Korean fir have been up-regulated after heat stress (Table 25). Therefore, the inductions of Hsps are critical for acclimating to heat stress.

This first comprehensive transcriptomic analysis of Korean fir provides a valuable genomic resource for further studies of other Abies species. Additionally, the present invention will provide important new insights into heat-stress adaptation and will facilitate further studies on Korean fir genes and their functions.

As a conclusion, the present invention represents a fully characterized transcriptome and provides valuable resources for genomic studies in Korean fir under heat stress.

The present invention can be described more concretely by following Examples.

### (Example 1) Plant Material and Treatments

Korean fir (Abies koreana Wilson) seeds were collected from Mount Halla on Jeju Island, Korea (33° 13-36′ N, 126° 12-57′ E). Seeds were sown in seedling trays with soil after breaking dormancy at 4° C. for three months. A single 1-year-old seedling was transplanted into each pot filled with same soil. Plants were grown in a greenhouse under natural sunlight conditions. The heat-stress treatment was performed on 3-year-old pot-growing plants in a growth chamber set to 30° C. under photoperiodic conditions (photon flux density of 180 μmol m⁻² s⁻¹). The 3-year-old seedlings were exposed to normal growth conditions (22° C.) and heat stress (30° C.), and then needles were harvested 21d after heat treatments.

### (Example 2) Library Preparation and RNA Sequencing

RNA samples were extracted from the needles of 21-d heat-treated and control plants. Total RNA was isolated using TRIzol reagent according to the manufacturers protocol (GibcoBRL, Cleveland, Ohio, USA). The RNA was analysed for quality and concentration using a 2100 Bioanalyzer (Agilent Technologies, Palo Alto, Calif., USA). A total of 3 μg of RNA for each sample was used in library construction with the Illumina Truseq RNA sample Preparation Kit (Illumina, Inc. San Diego, Calif., USA) per the manufacturers instructions. Briefly, mRNA was enriched using magnetic beads containing poly-T molecules. Following purification, the enriched mRNA was broken into small fragments. Random oligonucleotides and SuperScript II were used to synthesise the first-strand cDNA. The second-strand cDNA was subsequently synthesised using DNA Polymerase I and RNase H. Finally, end repair was carried out on these cDNA fragments, and they were ligated with Illumina adapters. Libraries were amplified using PCR according to Illumina guidelines. Libraries with insert sizes of 200 bp were constructed and then sequenced using the Illumina HiSeq 2000.

### (Example 3) De Novo Transcriptome Assembly and Annotation

Transcriptome assembly was accomplished using Trinity software, which first combined reads with certain lengths of overlap to form longer fragments without ambiguous bases, named as contigs. Contigs were then connected by Trinity to generate sequences that could not be extended on either end. These sequences were named as transcripts. Gene functions were annotated based on the NCBI non-redundant protein sequences and GO. A functional enrichment analysis of transcripts using the GO categories molecular functions, biological processes, and cellular components was performed using the Blast2GO program (version 2.5.0).

### (Example 4) Identification of DETs

Transcript expression levels were calculated using fragments per kb per million fragments method, which eliminated the influence of different gene lengths and sequencing levels. To isolate DETs with 2-fold higher or lower expressions of transcripts between control and heat-treated libraries, a rigorous algorithm developed based on a previous method was used.

### (Example 5) TFs and Hsp Analysis

TFs were predicted according to protein sequences obtained from coding sequence predictions. To search for the domains, we used plant TFs (http://pintfdb.bio.uni-potsdam.de/v3.0/) and classified transcripts according to the gene family's information. To identify the Hsps represented in our samples, transcript sequences were queried against the list of Hsp domain sequences from the HSRIP (http://pdslab.biochem.iisc.ernet.in/hspir) database. TransDecoder (http://transdecoder.sourceforge.net/) was used to predicate optimal open reading frame information with an 80-amino acid minimum protein length.

### (Example 6) qRT-PCR

In total, 14 DETs were selected to confirm that they were involved in responding to heat stress as assessed by qRT-PCR. Total RNAs (1 μg) of each sample were reverse transcribed using a Power cDNA Synthesis Kit (Intron Biotech Inc., Sungnam, Korea). The specific primers used for qRT-PCR are listed in Tables 7-9. qRT-PCR was carried out on a Bio-Rad CFX qRT-PCR detection system (Bio-Rad Laboratories Inc., CA, USA) using iQ™ SYBR® Green supermix (Bio-Rad). The reaction was performed under the following conditions: 95° C. for 10 min, followed by 45 cycles of 95° C. for 10 s and 60° C. for 30 s. The qRT-PCR reactions were repeated in three biological and three technical replications.

What is claimed is:

1. An isolated gene expressed in response to heat treatment of the Korean fir of Abies genus,
wherein the expression of an isolated gene of c142609_g1_i1 (NAC) (SEQ ID NO: 1); c207159_g1_i1 (MYB) (SEQ ID NO: 2); c124199_g1_i1 (ERF) (SEQ ID NO: 3); and c173884_g1_i1 (bHLH) (SEQ ID NO: 4) have been up-regulated,
the expression of an isolated gene of c85122_g1_i1 (MYB) (SEQ ID NO: 5); c199182_g1_i2 (bHLH) (SEQ ID NO: 6); and c189548_g3_i1 (ERF) (SEQ ID NO: 7) have been down-regulated.

2. An isolated gene that encoded HSP (heat shock protein) expressed in response to heat treatment of the Korean fir of Abies genus,
wherein the expression of an isolated gene of c217843_g2_i1 (Hsp90) (SEQ ID NO: 8); c149565_g1_i1 (Hsp70) (SEQ ID NO: 9); c199303_g3_i1 (Hsp60) (SEQ ID NO: 10); and c156586_g1_i1 (sHsp) (SEQ ID NO: 11) have been up-regulated,
the expression of an isolated gene of c205143_g5_i1 (Hsp90) (SEQ ID NO: 12); c149639_g1_i1 (Hsp70) (SEQ ID NO: 13); and c202543_g1_i1 (Hsp70) (SEQ ID NO: 14) have been down-regulated.

3. The isolated genes expressed in response to heat treatment of the Korean fir of Abies genus according to claim 1, wherein
a gene of c142609_g1_i1 (NAC) (SEQ ID NO: 1) has been isolated using the primer pair set of SEQ ID NO: 15 and SEQ ID NO: 16,
a gene of c207159_g1_i1 (MYB) (SEQ ID NO: 2); has been isolated using the primer pair set of SEQ ID NO: 17 and SEQ ID NO: 18,
a gene of c124199_g1_i1 (ERF) (SEQ ID NO: 3) has been isolated using the primer pair set of SEQ ID NO: 19 and SEQ ID NO: 20,
a gene of c173884_g1_i1 (bHLH) (SEQ ID NO: 4) has been isolated using the primer pair set of SEQ ID NO: 21 and SEQ ID NO: 22,
a gene of c85122_g1_i1 (MYB) (SEQ ID NO: 5) has been isolated using the primer pair set of SEQ ID NO: 23 and SEQ ID NO: 24,
a gene of c199182_g1_i2 (bHLH) (SEQ ID NO: 6) has been isolated using the primer pair set of SEQ ID NO: 25 and SEQ ID NO: 26 and
a gene of c189548_g3_i1 (ERF) (SEQ ID NO: 7) has been isolated using the primer pair set of SEQ ID NO: 27 and SEQ ID NO: 28.

4. The isolated genes that encoded HSP (heat shock protein) expressed in response to heat treatment of the Korean fir of Abies genus according to claim 2, wherein
a gene of c217843_g2_i1 (Hsp90) (SEQ ID NO: 8) has been isolated using the primer pair set of SEQ ID NO: 29 and SEQ ID NO: 30,
a gene of c149565_g1_i1 (Hsp70) (SEQ ID NO: 9) has been isolated using the primer pair set of SEQ ID NO: 31 and SEQ ID NO: 32,
a gene of c199303_g3_i1 (Hsp60) (SEQ ID NO: 10) has been isolated using the primer pair set of SEQ ID NO: 33 and SEQ ID NO: 34,
a gene of c156586_g1_i1 (sHsp) (SEQ ID NO: 11) has been isolated using the primer pair set of SEQ ID NO: 35 and SEQ ID NO: 36,
a gene of c205143_g5_i1 (Hsp90) (SEQ ID NO: 12) has been isolated using the primer pair set of SEQ ID NO: 37 and SEQ ID NO: 38,
a gene of c149639_g1_i1 (Hsp70) (SEQ ID NO: 13) has been isolated using the primer pair set of SEQ ID NO: 39 and SEQ ID NO: 40 and
a gene of c202543_g1_i1 (Hsp70) (SEQ ID NO: 14) has been isolated using the primer pair set of SEQ ID NO: 41 and SEQ ID NO: 42.
